(19) United States
(12) Patent Application Publication
(10) Pub. No.: US 2021/0253646 A1
(43) Pub. Date: Aug. 19, 2021

(54) VACCINE VECTOR ENCODING MUTATED GNAQ FOR TREATMENT OF UVEAL MELANOMA AND CANCERS HAVING ONCOGENIC MUTATIONS ON GNAQ AND GNA11 PROTEINS

(71) Applicant: THOMAS JEFFERSON UNIVERSITY, Philadelphia, PA (US)

(72) Inventors: Vitali Alexeev, Philadelphia, PA (US); Takami Sato, Wallingford, PA (US)

(21) Appl. No.: 17/252,123

(22) PCT Filed: Jun. 14, 2019
(86) PCT No.: PCT/US2019/037243

§ 371 Date: Dec. 14, 2020

Related U.S. Application Data
(60) Provisional application No. 62/685,171, filed on Jun. 14, 2018.

(51) Int. Cl. C07K 14/005; C07K 14/47; A61P 35/00; A61K 39/00; A61K 38/19; C07K 14/74

(52) U.S. Cl. CPC C07K 14/005; C07K 14/4722; A61P 35/00; A61K 39/0011; A61K 38/195; C07K 14/70539; A61K 2039/585; C12N 2710/16634; C12N 2710/16622; C12N 2710/16671; C07K 2319/00; A61K 2039/53; A61K 39/001114

(57) ABSTRACT

Provided is a composition comprising a mutant Q209L-GNAQ DNA vaccine encoding, in a N-terminal to C-terminal direction, a fusion protein comprising VP22 or an HLA-binding sequence thereof, a mutant GNAQ sequence comprising a Q209L mutation, and a PADRE epitope. Also provided are methods of treatment and methods of vaccination comprising administering to a patient the composition. Also provided is a method of generating mutant GNAQ-specific T cells comprising priming T cells with ex vivo cultured dendritic cells transduced or electroplated with the composition.

### CROSS REFERENCE TO RELATED APPLICATION

The present application is entitled to priority under 35 U.S.C. § 119(e) to U.S. Provisional Patent Application No. 62/685,171 filed Jun. 14, 2018, which is hereby incorporated by reference in its entirety herein.

### BACKGROUND OF THE INVENTION

Uveal melanoma is the most common intraocular malignancy in adults, representing 3.1% of all recorded cased of melanoma since 1970. The mean age-adjusted incidence of uveal melanoma in the United States is 5.1 per million, with the majority of cases (97.8%) occurred in the white population out of which more than 50% are represented by HLA-A2+ subjects. Accordingly, roughly 2,500 adults are diagnosed with ocular melanoma every year in the United States and a total of about 100 to 200 thousand uveal melanoma patients worldwide.

Despite improvements in the local treatment of the primary uveal melanoma (UM), there has been no change in the 5-year relative survival rate in the past three decades. Accordingly, patients having UM have a high mortality rate, as the disease is associated with the development and rapid progression of the metastatic disease. Of concern is that UM has a propensity to metastasize into the liver, and once it has reached the liver, mortality typically occurs within a few months. Indeed, 80% of metastatic patients die within 1 year and 92% die with 2 years, with mean survival at only a few months.

Presently, there is no active vaccination approach developed for uveal melanoma. There remains a need for compositions and methods for treating and preventing uveal melanoma.

### SUMMARY OF THE INVENTION

Provided is a composition comprising a mutant Q209L-GNAQ DNA vaccine encoding, in a N-terminal to C-terminal direction, a fusion protein comprising VP22 or an HLA-binding sequence thereof, a mutant GNAQ sequence comprising a Q209L mutation, and a PADRE epitope. In some embodiments, the mutant GNAQ sequence comprising a Q209L mutation is a full-length GNAQ sequence. In some embodiments, the mutant GNAQ sequence comprising a Q209L mutation is a short GNAQ sequence. In some embodiments, the mutant GNAQ sequence comprising a Q209L mutation comprises additional substitutions selected from the group consisting of V204P and V205L/E212V, wherein the addition substitutions improve binding of the fusion protein to HLA-A2 and enhance T cell activation. In some embodiments, the VP 22 is encoded by a nucleic acid sequence according to SEQ ID NO: 6 or 18. In further embodiments, the VP 22 is encoded by a nucleic acid sequence having at least 90%, at least 95%, or at least 99% homology to SEQ ID NO: 6 or 18. In some embodiments, the PADRE epitope is encoded by a nucleic acid sequence according to SEQ ID NO: 5. In further embodiments, the PADRE epitope is encoded by a nucleic acid sequence having at least 90%, at least 95%, or at least 99% homology to SEQ ID NO: 5. In some embodiments, the mutant Q209L-GNAQ DNA vaccine is encoded by a nucleic acid sequence according to SEQ ID NO: 12 or 14. In further embodiments, the mutant Q209L-GNAQ DNA vaccine is encoded by a nucleic acid sequence having at least 90%, at least 95%, or at least 99% homology to SEQ ID NO: 12 or 14. In some embodiments, the mutant Q209L-GNAQ DNA vaccine encodes a fusion protein comprising an amino acid sequence corresponding to SEQ ID NO: 3. In some embodiments, the mutant Q209L-GNAQ DNA vaccine encodes a fusion protein wherein the mutant GNAQ sequence comprising a Q209L mutation has at least 90%, at least 95%, or at least 99% homology to SEQ ID NO: 3. In some embodiments, the mutant GNAQ sequence comprising a Q209L mutation comprises at least 20 amino acids from Serine at position 198 to Isoleucine at position 217 of SEQ ID NO: 3. In further embodiments, the mutant GNAQ sequence comprising a Q209L mutation is encoded by a nucleic acid sequence comprising SEQ ID NO: 7 or 19.

Also provided is a method of treating an ocular cancer having a GNAQ or GNA11 mutation comprising: administering to a patient a composition comprising a mutant Q209L-GNAQ DNA vaccine encoding, in a N-terminal to C-terminal direction, a fusion protein comprising VP22 or an HLA-binding sequence thereof, a mutant GNAQ sequence comprising a Q209L mutation, and a PADRE epitope. In some embodiments, the mutant GNAQ sequence comprising a Q209L mutation is a full-length GNAQ sequence. In some embodiments, the mutant GNAQ sequence comprising a Q209L mutation is a short GNAQ sequence. In some embodiments, the VP 22 is encoded by a nucleic acid sequence having at least 90%, at least 95%, or at least 99% homology to SEQ ID NO: 6 or 18. In some embodiments, the PADRE epitope is encoded by a nucleic acid sequence having at least 90%, at least 95%, or at least 99% homology to SEQ ID NO: 5. In some embodiments, the mutant Q209L-GNAQ DNA vaccine is encoded by a nucleic acid sequence having at least 90%, at least 95%, or at least 99% homology to SEQ ID NO: 12 or 14.

In some embodiments, the mutant GNAQ sequence comprising a Q209L mutation comprises at least 20 amino acids from Serine at position 198 to Isoleucine at position 217 of SEQ ID NO: 3.

In some embodiments, the mutant GNAQ sequence comprising a Q209L mutation is encoded by a nucleic acid sequence comprising SEQ ID NO: 7 or 19.

In some embodiments, the method further comprises priming of the DNA vaccine administration site with a chemokine. In further embodiments, the chemokine is CCL21.

Provided is a method of generating a cytotoxic immune response against uveal melanoma comprising: administering to a patient a composition comprising a mutant Q209L-GNAQ DNA vaccine encoding, in a N-terminal to C-terminal direction, a fusion protein comprising VP22 or an HLA-binding sequence thereof, a mutant GNAQ sequence comprising a Q209L mutation, and a PADRE epitope. In some embodiments, the mutant GNAQ sequence comprising a Q209L mutation is a full-length GNAQ sequence. In some embodiments, the mutant GNAQ sequence comprising a Q209L mutation is a short GNAQ sequence. In some embodiments, the VP 22 is encoded by a nucleic acid sequence having at least 90%, at least 95%, or at least 99% homology to SEQ ID NO: 6 or 18. In some embodiments, the PADRE epitope is encoded by a nucleic acid sequence having at least 90%, at least 95%, or at least 99% homology to SEQ ID NO: 5. In some embodiments, the mutant Q209L-GNAQ DNA vaccine is encoded by a nucleic acid sequence having at least 90%, at least 95%, or at least 99% homology to SEQ ID NO: 12 or 14.

In some embodiments, the mutant GNAQ sequence comprising a Q209L mutation comprises at least 20 amino acids from Serine at position 198 to Isoleucine at position 217 of SEQ ID NO: 3.

In some embodiments, the mutant GNAQ sequence comprising a Q209L mutation is encoded by a nucleic acid sequence comprising SEQ ID NO: 7 or 19.

Provided is a method of providing prophylactic vaccination of a high risk patient after treatment of primary intraocular lesions comprising malignant cells harboring Q209L mutated GNAQ or GNA11 cells, said vaccination comprising: administering to a patient a composition comprising a mutant Q209L-GNAQ DNA vaccine encoding, in a N-terminal to C-terminal direction, a fusion protein comprising VP22 or an HLA-binding sequence thereof, a mutant GNAQ sequence comprising a Q209L mutation, and a PADRE epitope. In some embodiments, the mutant GNAQ sequence comprising a Q209L mutation is a full-length GNAQ sequence. In some embodiments, the mutant GNAQ sequence comprising a Q209L mutation is a short GNAQ sequence. In some embodiments, the VP 22 is encoded by a nucleic acid sequence having at least 90%, at least 95%, or at least 99% homology to SEQ ID NO: 6 or 18. In some embodiments, the PADRE epitope is encoded by a nucleic acid sequence having at least 90%, at least 95%, or at least 99% homology to SEQ ID NO: 5. In some embodiments, the mutant Q209L-GNAQ DNA vaccine is encoded by a nucleic acid sequence having at least 90%, at least 95%, or at least 99% homology to SEQ ID NO: 12 or 14.

In some embodiments, the mutant GNAQ sequence comprising a Q209L mutation comprises at least 20 amino acids from Serine at position 198 to Isoleucine at position 217 of SEQ ID NO: 3.

In some embodiments, the mutant GNAQ sequence comprising a Q209L mutation is encoded by a nucleic acid sequence comprising SEQ ID NO: 7 or 19.

In some embodiments, the method further comprises priming of the DNA vaccine administration site with a chemokine. In further embodiments, the chemokine is CCL21.

In some embodiments, the mutant Q209L-GNAQ DNA vaccine encodes a fusion protein comprising an amino acid sequence corresponding to SEQ ID NO: 3.

In some embodiments, the mutant Q209L-GNAQ DNA vaccine encodes a fusion protein wherein the mutant GNAQ sequence comprising a Q209L mutation has at least 90%, at least 95%, or at least 99% homology to SEQ ID NO: 3.

In some embodiments, the mutant Q209L-GNAQ sequence comprises at least 20 amino acids from Serine at position 198 to Isoleucine at position 217 of SEQ ID NO: 3.

Provided is a method of therapeutic vaccination of patients with metastatic disease comprising malignant cells harboring Q209L mutated GNAQ or GNA11 cells, said vaccination comprising: administering to a patient a composition comprising a mutant Q209L-GNAQ DNA vaccine encoding, in a N-terminal to C-terminal direction, a fusion protein comprising VP22 or an HLA-binding sequence thereof, a mutant GNAQ sequence comprising a Q209L mutation, and a PADRE epitope. In some embodiments, the mutant GNAQ sequence comprising a Q209L mutation is a full-length GNAQ sequence. In some embodiments, the mutant GNAQ sequence comprising a Q209L mutation is a short GNAQ sequence. In some embodiments, the VP 22 is encoded by a nucleic acid sequence having at least 90%, at least 95%, or at least 99% homology to SEQ ID NO: 6 or 18. In some embodiments, the PADRE epitope is encoded by a nucleic acid sequence having at least 90%, at least 95%, or at least 99% homology to SEQ ID NO: 5. In some embodiments, the mutant Q209L-GNAQ DNA vaccine is encoded by a nucleic acid sequence having at least 90%, at least 95%, or at least 99% homology to SEQ ID NO: 12 or 14.

In some embodiments, the mutant GNAQ sequence comprising a Q209L mutation comprises at least 20 amino acids from Serine at position 198 to Isoleucine at position 217 of SEQ ID NO: 3.

In some embodiments, the mutant GNAQ sequence comprising a Q209L mutation is encoded by a nucleic acid sequence comprising SEQ ID NO: 7 or 19.

In some embodiments, the method further comprises priming of the DNA vaccine administration site with a chemokine. In further embodiments, the chemokine is CCL21.

In some embodiments, the mutant Q209L-GNAQ DNA vaccine encodes a fusion protein comprising an amino acid sequence corresponding to SEQ ID NO: 3.

In some embodiments, the mutant Q209L-GNAQ DNA vaccine encodes a fusion protein wherein the mutant GNAQ sequence comprising a Q209L mutation has at least 90%, at least 95%, or at least 99% homology to SEQ ID NO: 3.

In some embodiments, the mutant GNAQ sequence comprising a Q209L mutation comprises at least 20 amino acids from Serine at position 198 to Isoleucine at position 217 of SEQ ID NO: 3.

Provided is a method of vaccinating a mammal comprising administering to the mammal a composition comprising a mutant Q209L-GNAQ DNA vaccine encoding, in a N-terminal to C-terminal direction, a fusion protein comprising VP22 or an HLA-binding sequence thereof, a mutant GNAQ sequence comprising a Q209L mutation, and a PADRE epitope. In some embodiments, the mutant GNAQ sequence comprising a Q209L mutation is a full-length GNAQ sequence. In some embodiments, the mutant GNAQ sequence comprising a Q209L mutation is a short GNAQ sequence. In some embodiments, the mammal is human. In some embodiments, the mutant GNAQ sequence comprising a Q209L mutation is human. In some embodiments, the method comprises pre-treatment of a vaccine administration site with CCL21. In some embodiments, the VP 22 is encoded by a nucleic acid sequence having at least 90%, at least 95%, or at least 99% homology to SEQ ID NO: 6 or 18. In some embodiments, the PADRE epitope is encoded by a nucleic acid sequence having at least 90%, at least 95%, or at least 99% homology to SEQ ID NO: 5. In some embodiments, the mutant Q209L-GNAQ DNA vaccine is encoded by a nucleic acid sequence having at least 90%, at least 95%, or at least 99% homology to SEQ ID NO: 12 or 14.

In some embodiments, the mutant GNAQ sequence comprising a Q209L mutation comprises at least 20 amino acids from Serine at position 198 to Isoleucine at position 217 of SEQ ID NO: 3.

In some embodiments, the mutant GNAQ sequence comprising a Q209L mutation is encoded by a nucleic acid sequence comprising SEQ ID NO: 7 or 19.

In further aspects of any one of the previous embodiments, the nucleic acid sequence encoding a fusion protein comprising VP22 or an HLA-binding sequence thereof, a mutant GNAQ sequence comprising a Q209L mutation, and a PADRE epitope is linked with hCCL21 via a P2A ribosome skipping element allowing expression of both transgenes in one cell.

Provided is a method of creating a vaccine comprising generating a DNA encoding a fusion protein comprising a portion of an antigen and a portion of VP22 comprising regions which are enriched with HLA-A1 (amino acids 10-70), HLA-A2 (amino acids 210-260) and/or HLA-A3(amino acids 167-208) binding peptides of SEQ ID NO: 8. Also provided is a vaccine comprising DNA encoding a fusion protein comprising a portion of an antigen and a portion of VP22 comprising regions which are enriched with HLA-A1 (amino acids 10-70), HLA-A2 (amino acids 210-260) and/or HLA-A3(amino acids 167-208) binding peptides of SEQ ID NO: 8.

Also provided is a method of generating mutant Q209L-GNAQ-specific T cells comprising priming T cells with ex vivo cultured dendritic cells transduced or electroporated with the composition of any one of the previous embodiments.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 depicts that protein sequences for human GNAQ, human GNA11 and mouse GNAQ are highly homologous and identical in the region spanning a Q209L mutation in mutant human GNAQ. FIG. 1 specifies SEQ ID NO: 1 for human GNAQ, SEQ ID NO: 2 for hGNA11, SEQ ID NO: 3 for human GNAQ containing a Q209L mutation, and SEQ ID NO: 4 for mouse GNAQ.

FIG. 2 depicts results of the in silico analysis predictive of the mutated (Q209L) GNAQ peptide binding to mouse (H2-Kb) and human (HLA-A2.1) MHC class I. Wild type sequence is SEQ ID NO: 9. Mutant sequence is SEQ ID NO: 10.

FIG. 3 depicts fluorescence intensity observed in the in vitro MHC stabilization assay with wild type and mutated GNAQ peptides showing a more efficient binding of the Q209L peptides to mouse (H2/Kb) and human (HLA-A2.1) MHC. Mouse H2-Kb+RMAS and human HLA-A2+T2 cells were loaded with wild type and mutant GNAQ peptides. Data is presented as mean fluorescence intensity±SD from 6 independent experiments.

FIG. 4A depicts a full length polypeptide sequence of the PADRE epitope (SEQ ID NO: 11).

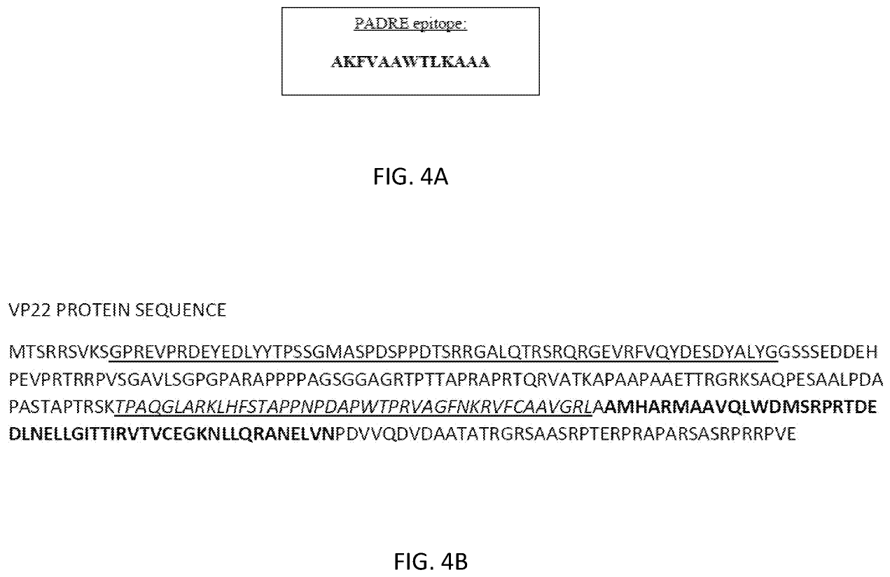

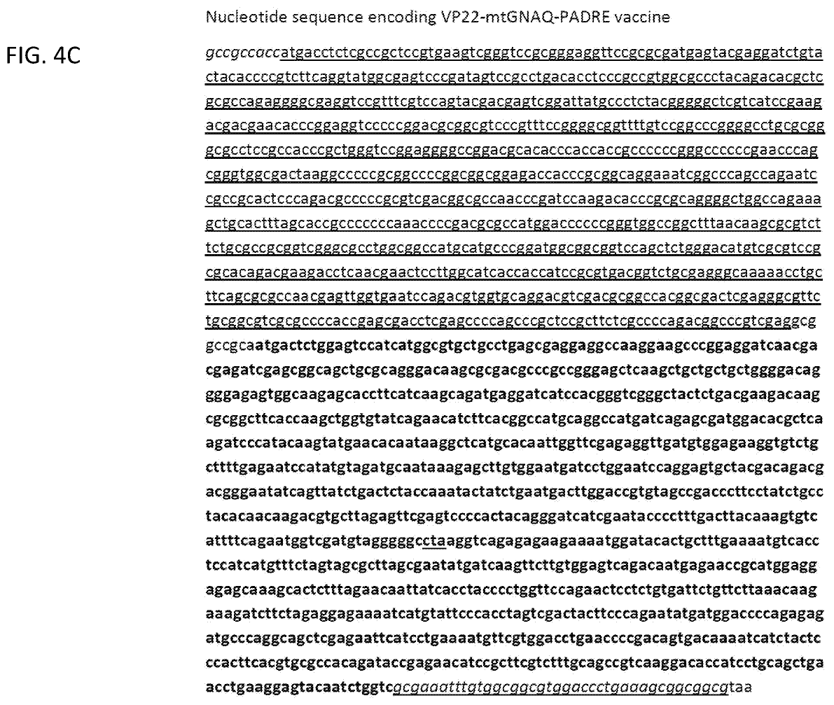

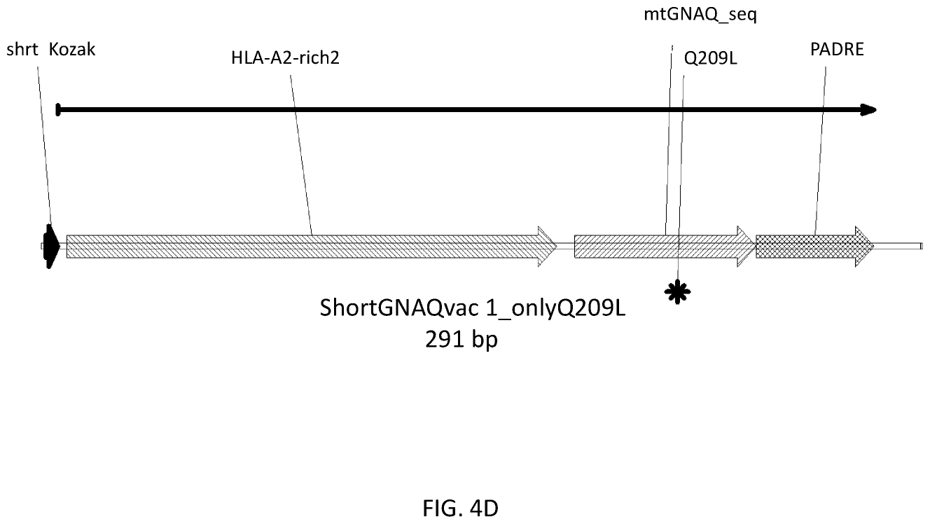

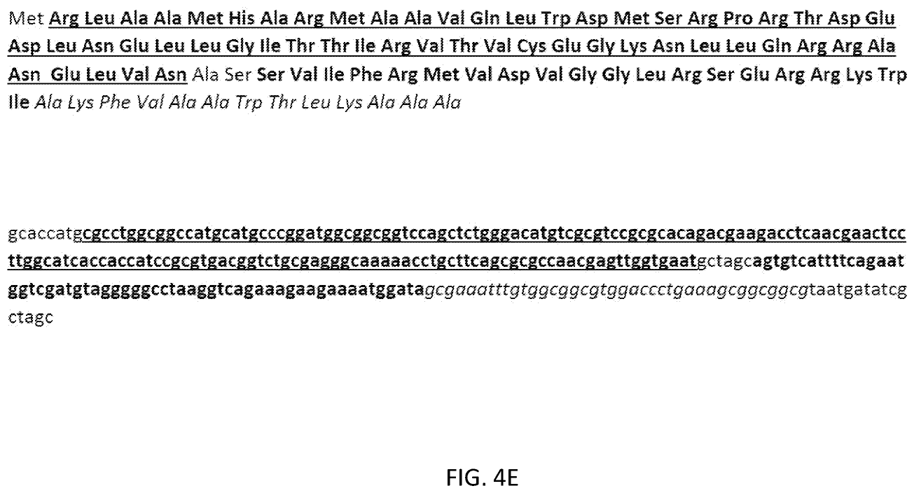

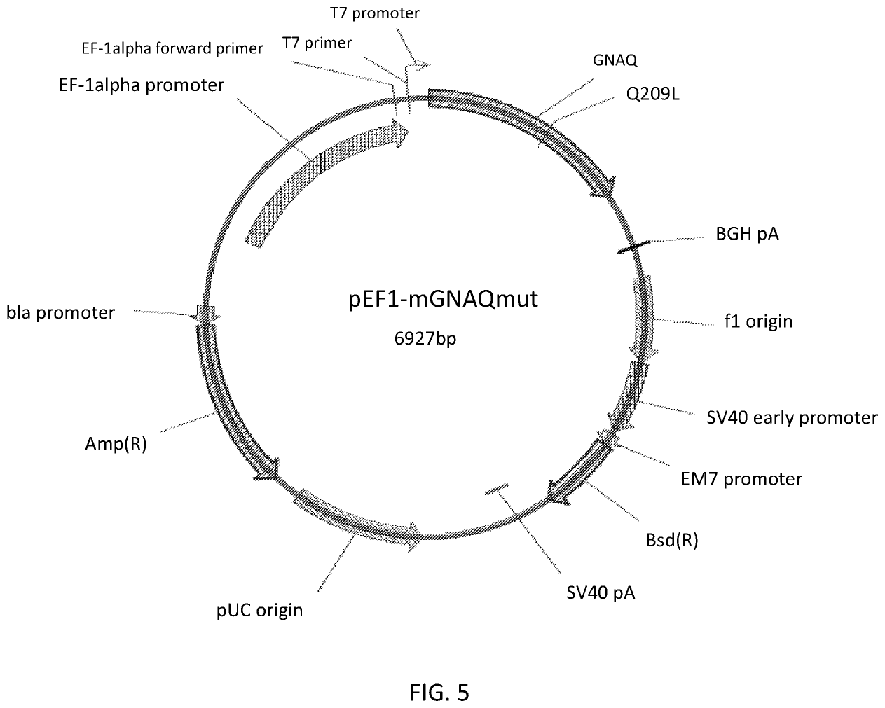

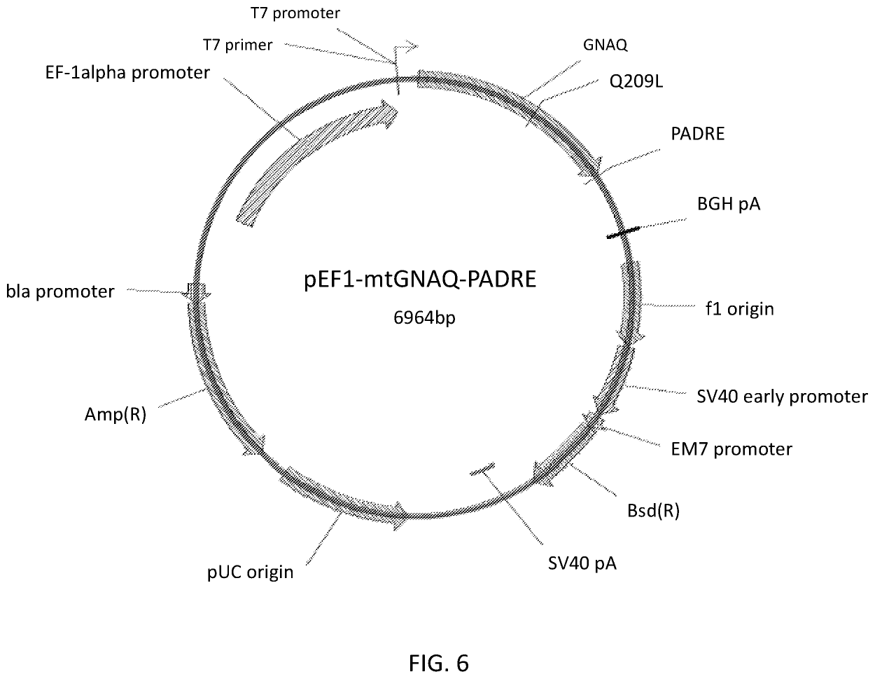

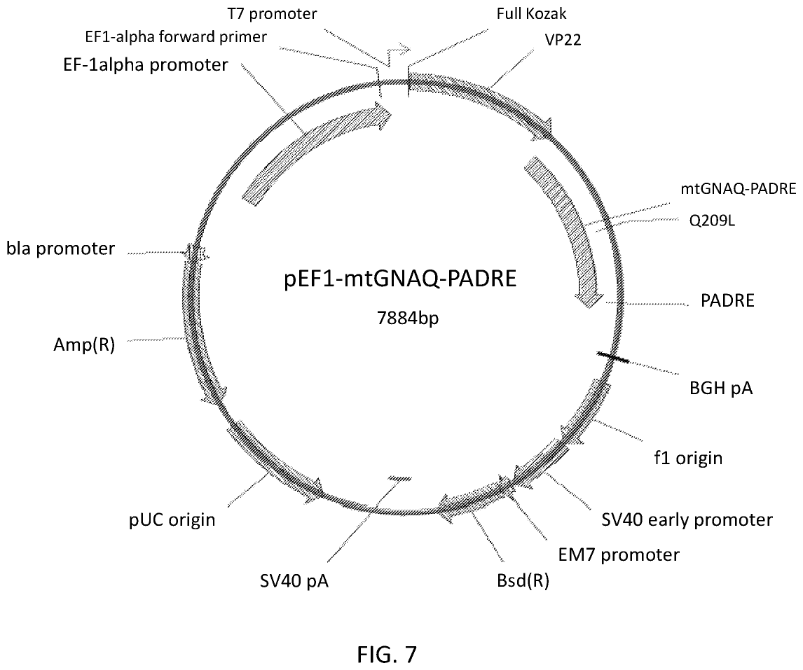

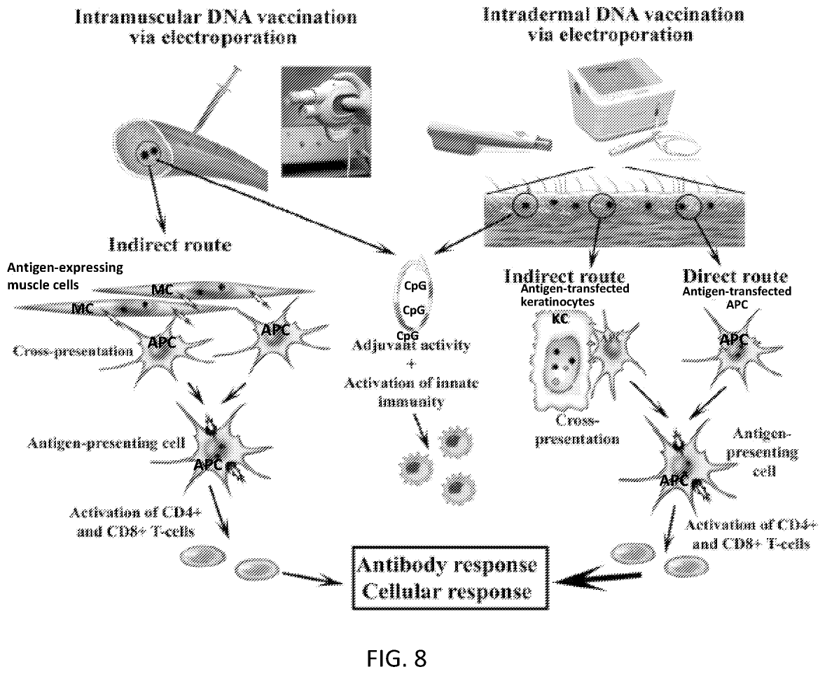

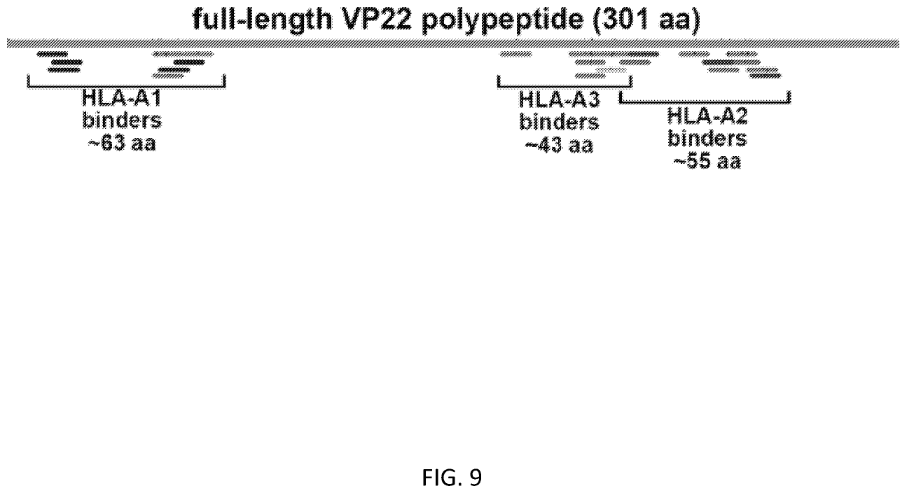

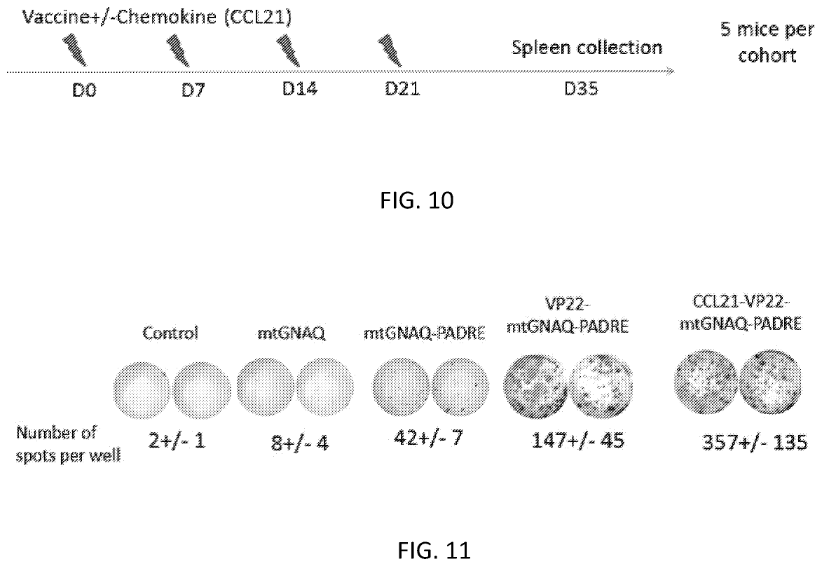

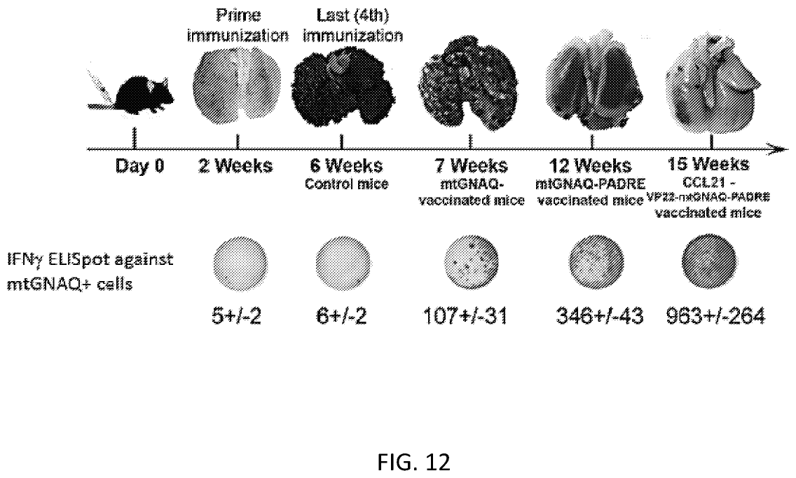

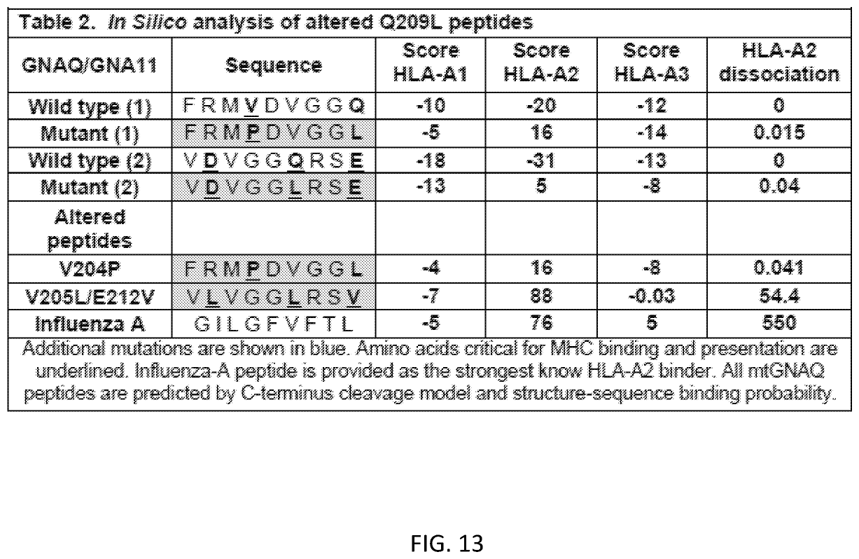

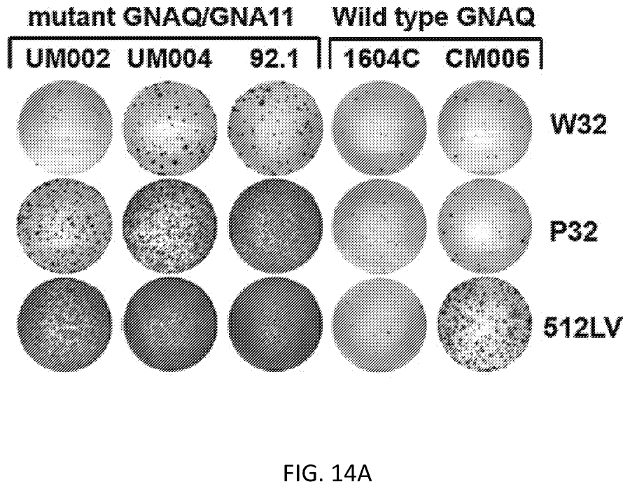

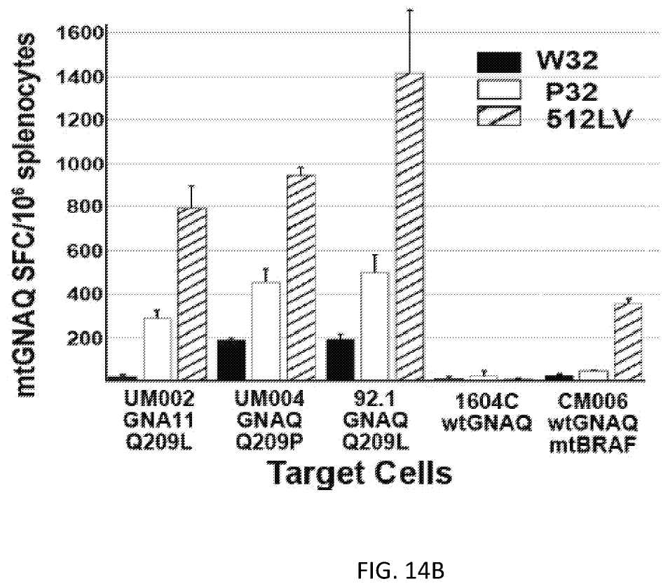

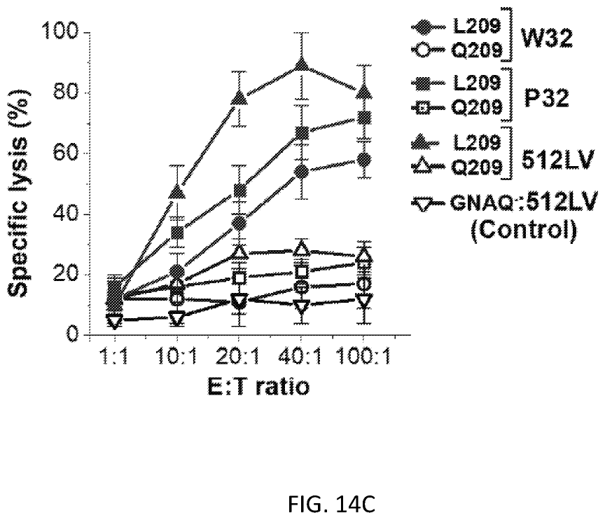

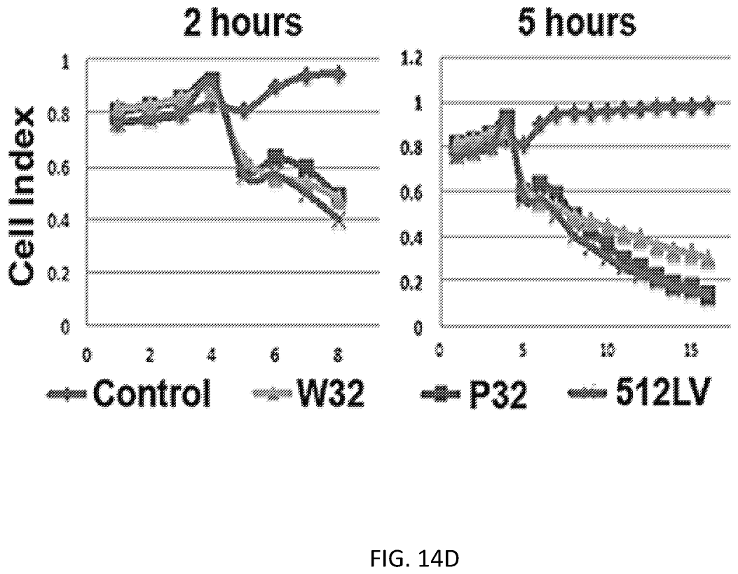

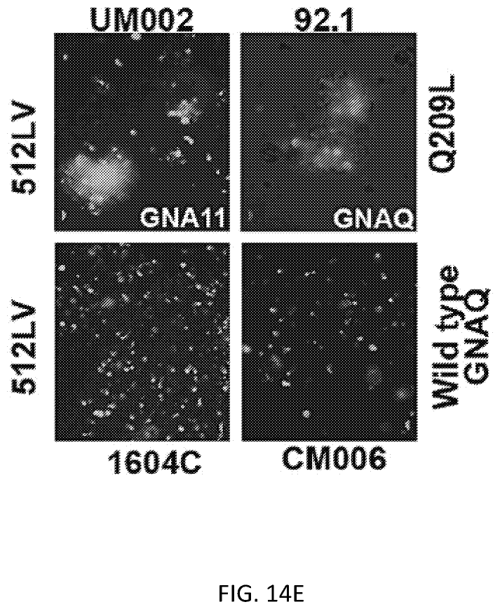

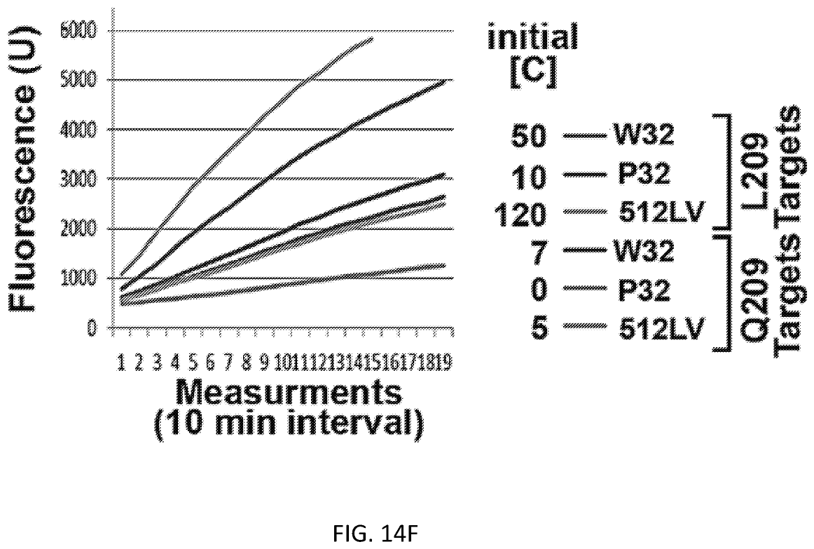

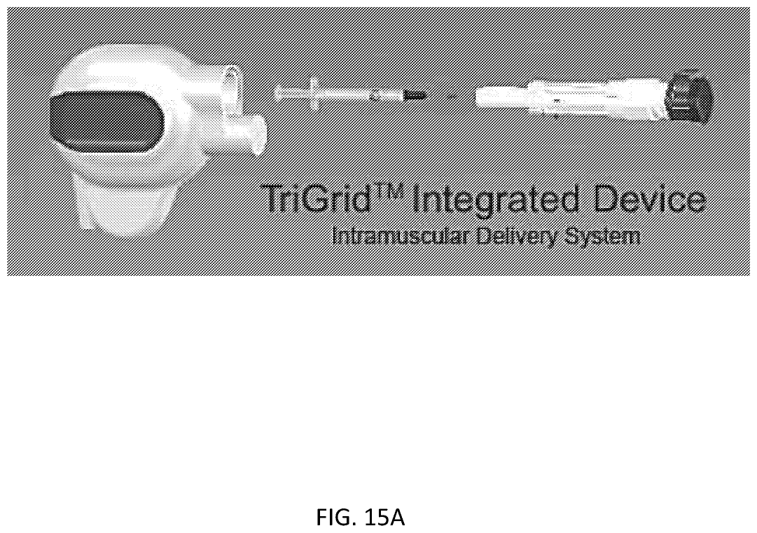

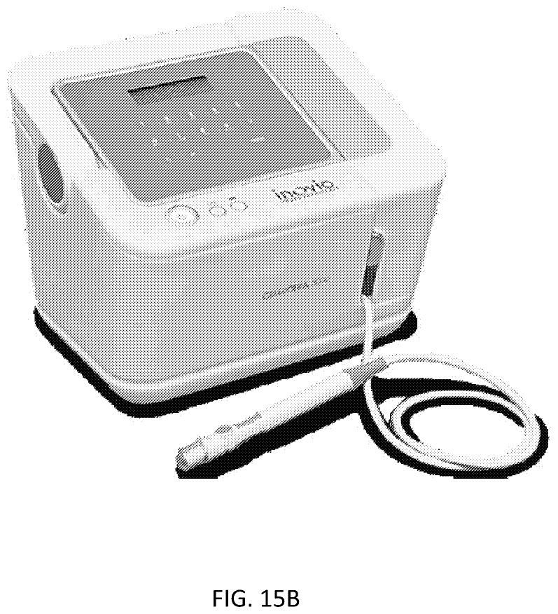

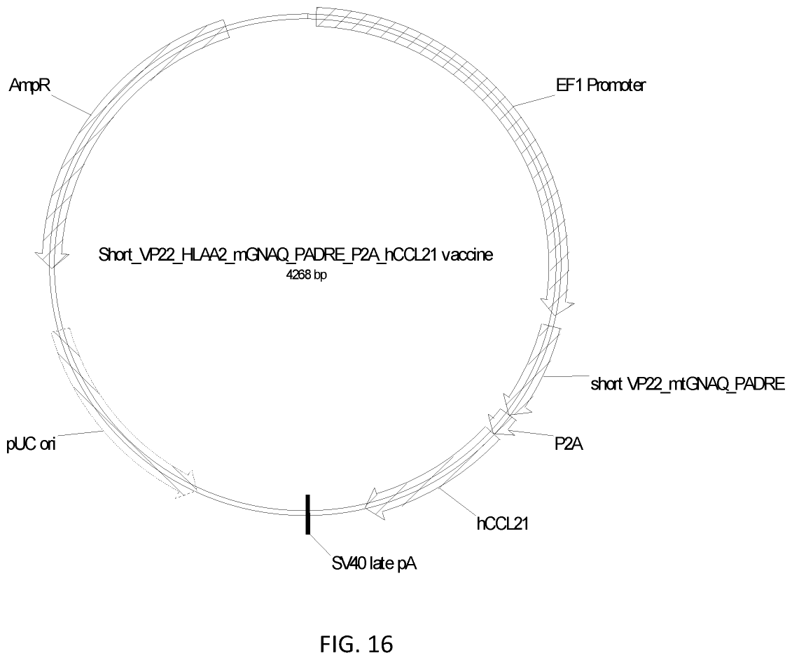

FIG. 4B depicts a full-length polypeptide sequence of the VP22 protein (SEQ ID NO: 8) with highlighted regions enriched with HLA-A1 (underlined), HLA-A2 (bold) and HLA-A3 (italicized and underlined) binding peptides.
Underlined sequence—region enriched with HLA-A1 binders;
Italicized and underlined sequence—region enriched with HLA-A3 binders
Bolded sequence—region enriched with HLA-A2 binders

FIG. 4C depicts a full-length DNA sequence encoding VP22-mtGNAQ-PADRE fusion protein (SEQ ID NO: 12). The PADRE sequence is SEQ ID No. 5; the VP22 Sequence is SEQ ID NO: 6; and the mtGNAQ full length sequence is SEQ ID NO: 7.
Italicized sequence—Full-length Kozak sequence
Underlined sequence—DNA sequence encoding full-length VP22 (SEQ ID NO: 6)
Bolded sequence—Full-length mutant mtGNAQ (SEQ ID NO: 7)
Bolded and underlined—GNAQ mutation site (mutant triplet resulting in Q209L tumorigenic mutation)
Italicized and underlined sequence—PADRE epitope (SEQ ID NO: 5)

FIG. 4D depicts a vaccine comprising a short VP22 sequence, a short sequence of GNAQ containing Q209L mutation and PADRE epitopes.

FIG. 4E depicts a sequence encoding a short fusion vaccine (depicted in FIG. 4D) amino acid sequence (SEQ ID NO: 13) and nucleic acid sequence (SEQ ID NO: 14). The VP22 amino acid sequence is SEQ ID NO: 15 (bold/underlined); the GNAQ amino acid sequence is SEQ ID NO: 16 (bold) and the PADRE amino acid sequence is SEQ ID NO: 17 (italics).
Bolded and Underlined sequence—HLA-A2 enriched VP22 sequence (SEQ ID NO: 15)
Bolded sequence—short sequence of the mtGNAQ-encoding cDNA containing a sequence encoding Q209L mutation (SEQ ID NO: 16)
Italicized sequence—sequence encoding PADRE epitope (SEQ ID NO: 17)

The VP22 nucleic acid sequence is SEQ ID NO: 18 (bold/underlined); the GNAQ nucleic acid sequence is SEQ ID NO: 19 (bold) and the PADRE nucleic acid sequence is SEQ ID NO: 5 (italicized).
Bolded and Underlined sequence—HLA-A2 enriched VP22 sequence (SEQ ID NO: 18)
Bolded sequence—short sequence of the mtGNAQ-encoding cDNA containing a sequence encoding Q209L mutation (SEQ ID NO: 19)
Italicized sequence—sequence encoding PADRE epitope (SEQ ID NO: 5)

FIG. 5 depicts the DNA Vaccine design; pEF1-mtGNAQ contains a full-length mutant GNAQ (Q209L) sequence.

FIG. 6 depicts pEF1-mtGNAQ-PADRE containing a full-length mutant GNAQ (Q209L) sequence fused in frame on the 3′ end with pan HLA DR-binding epitope PADRE (T-helper epitope), which activates T-helper cells.

FIG. 7 depicts pEF1-VP22-mtGNAQ-PADRE containing a full-length mutant GNAQ (Q209L)-PADRE fused in frame with the full-length VP22 protein on the 5′ end. The Herpes simplex virus 1 VP22 activates cytotoxic T cells.

FIG. 8 depicts a flowchart of the DNA vaccination via intramuscular and intradermal electroporation which leads to the development of the cytotoxic and humoral immunity via direct and indirect routes of antigen presentation.

FIG. 9 depicts a VP 22 protein sequence with predicted HLA-A1, HLA-A2.1 and HLA-A3—binding peptides outlining 3 HLA-binding regions. Sequences encoding these regions can be used in place of the full length VP22-encoding sequence to customize vaccine.

FIG. 10 depicts prophylactic vaccination including priming of the vaccine administration site with chemokine(s) following in vivo electroporation of a plasmid DNA (DNA vaccine).

FIG. 11 depicts an IFNγ ELISpot assay showing reactivity of splenocytes isolated from differently vaccinated mice as effectors and mtGNAQ+ cells as targets. Spots of the IFNγ ELISpot against mtGNAQ+ cells reflect T cell activation. A vaccine comprised of VP22-mtGNAQ-PADRE was most effective in activating mtGNAQ specific T cell response. Priming of the vaccine administration site with CCL21 additionally enhanced T cell activation.

FIG. 12 depicts pulmonary tumor burden in differently vaccinated mice at various time points (top images). The bottom images show IFNγ ELISPOT against mtGNAQ+ cells using splenocytes isolated from differently vaccinated mice as effectors. Vaccination with VP22-mtGNAQ-PADRE led to the robust T cell activation and inhibition of metastatic lesions.

FIG. 13 depicts results of the in silico analysis showing that additional alteration to the mutant (Q209L) GNAQ peptides could enhance peptide binding to MHC class I, specifically HLA-A2.1 and stability of the MHC-peptide complex (provided for HLA-A2.1). Wild type (1) amino acid sequence is SEQ ID NO: 20; Mutant (1) amino acid sequence is SEQ ID NO: 21; Wild Type (2) amino acid sequence is SEQ ID NO: 22; Mutant (2) amino acid sequence is SEQ ID NO: 23; V204P amino acid sequence is SEQ ID NO: 24; V205L/E212V amino acid sequence is SEQ ID NO: 25; Influenza A amino acid sequence is SEQ ID NO: 26.

FIGS. 14A-14F depicts that introduction of additional alterations to the mtGNAQ sequence in the DNA vaccine enhances activation of human T cells in vitro. FIG. 14A depicts T cell activation after in vitro priming with different vaccine vectors as determined by IFNγ ELISpot assay; FIG. 14B depicts quantitation of the spot-forming cells shown on FIG. 14A; FIG. 14C depicts cytotoxic activity of differently primed T cells against wild type (Q209) and mutated (L209) target cells; FIG. 14D depicts short term cytotoxic activity of differently primed T cells; FIG. 14E depicts direct binding of activated T cells (green) to wild type and mutant GNAQ targets; FIG. 14F depicts Granzyme B activity of differently primed T cells after 2 h of exposure to wild type and mutant GNAQ targets.

FIGS. 15A-15B depict in vivo electroporation devices, as non-limiting examples of devices suitable for in vivo electroporation as described in the methods herein.

FIG. 16 is a diagram depicting a vaccine plasmid design comprising short VP22, short mtGNAQ, and PADRE epitopes. The nucleic acid sequence encoding a short VP22, short mtGNAQ, and PADRE epitope fusion protein (depicted in FIG. 4E) is linked with hCCL21 via a P2A ribosome skipping element allowing expression of both transgenes in one cell.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

The term “antibody” or “Ab” as used herein, refers to a protein, or polypeptide sequence derived from an immunoglobulin molecule, which specifically binds to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv) and humanized antibodies (Harlow et al., 1998, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). An antibody may be derived from natural sources or from recombinant sources. Antibodies are typically tetramers of immunoglobulin molecules.

The term “ameliorating” or “treating” means that the clinical signs and/or the symptoms associated with a disease are lessened as a result of the actions performed. The signs or symptoms to be monitored will be well known to the skilled clinician.

As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The term “biological” or “biological sample” refers to a sample obtained from an organism or from components (e.g., cells) of an organism. The sample may be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

As used herein, the terms “control,” or “reference” are used interchangeably and refer to a value that is used as a standard of comparison.

The term “immunogenicity” as used herein, refers to the innate ability of an antigen or organism to elicit an immune response in an animal when the antigen or organism is administered to the animal. Thus, “enhancing the immunogenicity” refers to increasing the ability of an antigen or organism to elicit an immune response in an animal when the antigen or organism is administered to an animal. The increased ability of an antigen or organism to elicit an immune response can be measured by, among other things, a greater number of antibodies that bind to an antigen or organism, a greater diversity of antibodies to an antigen or organism, a greater number of T-cells specific for an antigen or organism, a greater cytotoxic or helper T-cell response to an antigen or organism, a greater expression of cytokines in response to an antigen, and the like.

As used herein, the terms “eliciting an immune response” or “immunizing” refer to the process of generating a B cell and/or a T cell response against a heterologous protein.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Heterologous antigens” used herein to refer to an antigen that is not endogenous to the organism comprising or expressing an antigen. As an example, a virus vaccine vector comprising or expressing a viral or tumor antigen comprises a heterologous antigen. The term “Heterologous protein” as used herein refers to a protein that elicits a beneficial immune response in a subject (i.e. mammal), irrespective of its source.

The term “specifically binds”, “selectively binds” or “binding specificity” refers to the ability of the humanized antibodies or binding compounds of the invention to bind to a target epitope with a greater affinity than that which results when bound to a non-target epitope. In certain embodiments, specific binding refers to binding to a target with an affinity that is at least 10, 50, 100, 250, 500, or 1000 times greater than the affinity for a non-target epitope.

As used herein, by “combination therapy” is meant that a first agent is administered in conjunction with another agent. “In combination with” or “In conjunction with” refers to administration of one treatment modality in addition to another treatment modality. As such, “in combination with” refers to administration of one treatment modality before, during, or after delivery of the other treatment modality to the individual. Such combinations are considered to be part of a single treatment regimen or regime.

“Humoral immunity” or “humoral immune response” both refer to B-cell mediated immunity and are mediated by highly specific antibodies, produced and secreted by B-lymphocytes (B-cells).

“Prevention” refers to the use of a pharmaceutical compositions for the vaccination against a disorder.

“Adjuvant” refers to a substance that is capable of potentiating the immunogenicity of an antigen. Adjuvants can be one substance or a mixture of substances and function by acting directly on the immune system or by providing a slow release of an antigen. Examples of adjuvants are aluminium salts, polyanions, bacterial glycopeptides and slow release agents as Freund's incomplete.

“Delivery vehicle” refers to a composition that helps to target the antigen to specific cells and to facilitate the effective recognition of an antigen by the immune system. The best-known delivery vehicles are liposomes, virosomes, microparticles including microspheres and nanospheres, polymers, bacterial ghosts, bacterial polysaccharides, attenuated bacteria, virus like particles, attenuated viruses and ISCOMS.

As used herein, the term “expression cassette” means a nucleic acid sequence capable of directing the transcription and/or translation of a heterologous coding sequence. In some embodiments, the expression cassette comprises a promoter sequence operably linked to a sequence encoding a heterologous protein. In some embodiments, the expression cassette further comprises at least one regulatory sequence operably linked to the sequence encoding the heterologous protein.

“Incorporated into” or “encapsulated in” refers to an antigenic peptide that is within a delivery vehicle, such as microparticles, bacterial ghosts, attenuated bacteria, virus like particles, attenuated viruses, ISCOMs, liposomes and preferably virosomes.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “fusion protein” as used herein refers to a protein wherein the protein comprises two or more proteins linked together by peptide bonds or other chemical bonds.

The proteins can be linked together directly by a peptide or other chemical bond, or with one or more amino acids between the two or more proteins, referred to herein as a spacer.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “RNA” as used herein is defined as ribonucleic acid.

“Transform”, “transforming”, and “transformation” is used herein to refer to a process of introducing an isolated nucleic acid into the interior of an organism.

The term “treatment” as used within the context of the present invention is meant to include therapeutic treatment as well as prophylactic, or suppressive measures for the disease or disorder. As used herein, the term “treatment” and associated terms such as “treat” and “treating” means the reduction of the progression, severity and/or duration of a disease condition or at least one symptom thereof. The term ‘treatment’ therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

The term “equivalent,” when used in reference to nucleotide sequences, is understood to refer to nucleotide sequences encoding functionally equivalent polypeptides. Equivalent nucleotide sequences will include sequences that differ by one or more nucleotide substitutions, additions- or deletions, such as allelic variants; and will, therefore, include sequences that differ from the nucleotide sequence of the nucleic acids described herein due to the degeneracy of the genetic code.

The term “isolated” as used herein with respect to nucleic acids, such as DNA or RNA, refers to molecules separated from other DNAs or RNAs, respectively that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments, which are not naturally occurring as fragments and would not be found in the natural state. The term “isolated” is also used herein to refer to polypeptides, which are isolated from other cellular proteins and is meant to encompass both purified and recombinant polypeptides. An “isolated cell” or “isolated population of cells” is a cell or population of cells that is not present in its natural environment.

A “mutation” as used therein is a change in a DNA sequence resulting in an alteration from its natural state. The mutation can comprise a deletion and/or insertion and/or duplication and/or substitution of at least one deoxyribonucleic acid base such as a purine (adenine and/or thymine) and/or a pyrimidine (guanine and/or cytosine). Mutations may or may not produce discernible changes in the observable characteristics (phenotype) of an organism.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. There are numerous expression control sequences, including promoters which are native, constitutive, inducible and/or tissue-specific, are known in the art that may be used in the compositions of the invention. “Operably linked” should be construed to include RNA expression and control sequences in addition to DNA expression and control sequences.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence, which is required for expression of a gene product operably linked to the promoter/regulatory sequence. In some instances, this sequence may be the core promoter sequence and in other instances, this sequence may also include an enhancer sequence and other regulatory elements, which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

As used herein, the term “pharmaceutical composition” refers to a mixture of at least one compound useful within the invention with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the term “effective amount” or “therapeutically effective amount” means the amount of the virus like particle generated from vector of the invention which is required to prevent the particular disease condition, or which reduces the severity of and/or ameliorates the disease condition or at least one symptom thereof or condition associated therewith.

A “subject” or “patient,” as used therein, may be a human or non-human mammal. Non-human mammals include, for example, livestock and pets, such as ovine, bovine, porcine, canine, feline and murine mammals. Preferably, the subject is human.

A “vector” is a composition of matter which comprises an isolated nucleic acid and which can be used to deliver the isolated nucleic acid to the interior of a cell.

Numerous vectors are known in the art including, but not limited to, linear polynucleotides, polynucleotides associated with ionic or amphiphilic compounds, plasmids, and viruses. In the present disclosure, the term “vector” includes an autonomously replicating virus.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

Provided is a composition comprising a mutant Q209L-GNAQ DNA vaccine encoding, in a N-terminal to C-terminal direction, a fusion protein comprising VP22 or an HLA-binding sequence thereof, a mutant GNAQ sequence comprising a Q209L mutation, and a PADRE epitope. The fusion protein may comprise a full-length mutant GNAQ-encoding sequence or a short sequence spanning the Q209L mutation site.

Additional substitutions such as V204P or V205L/E212V may be introduced into the mutant GNAQ sequence to improve binding of the Q209L-harboring peptides(s) to enhance T cell trimming and activation.

In some embodiments, the DNA vaccine administration site is primed with a chemokine, e.g., CCL21.

In some embodiments, the mutant GNAQ sequence comprising a Q209L mutation is human.

Accordingly, a preferred embodiment is directed towards a method of generating a cytotoxic immune response against uveal melanoma using DNA vaccine comprising of fusion of sequences encoding VP22 or its HLA-binding sequence(s) at the 5′ end of mutated (Q209L) GNAQ and PADRE epitope at the 3′ end.

In a further embodiment, a method comprises prophylactic DNA vaccination of a high risk patient after treatment of primary intraocular lesions, or therapeutic DNA vaccination of patients with metastatic disease, comprising of malignant cells harboring Q209L mutated GNAQ or GNA11 cells. In preferred embodiments, the method comprises administering to a patient in need thereof a mutant Q209L-GNAQ DNA vaccine encoding, in a N-terminal to C-terminal direction, a fusion protein comprising VP22 or an HLA-binding sequence thereof, a mutant full-length or short GNAQ sequence comprising a Q209L mutation, and a PADRE epitope. A short mutant GNAQ-encoding sequence is illustrated in FIG. 4E. The DNA vaccine is designed to activate T cells, which specifically recognize mutated Q209L-harboring GNAQ/GNA11 peptides which are presented on the surface of malignant cells and MHC class I molecules, such as HLA-A2.

In preferred embodiments, the vaccination protocol comprises a step of pre-conditioning of the vaccine administration site with secondary lymphoid chemokine CCL21 to recruit leukocytes prior to DNA vaccination. Based on the current data and prior findings (Igoucheva, O., Grazzini, M., Pidich, A., Kemp, D., Larijani, M., Farber, M., Lorton, J., Rodeck, U., Alexeev, V.: Immunotargeting and eradication of orthotropic melanoma using a chemokine-enhanced DNA vaccine. Gene Therapy 2013 September; 20(9):939-48), such pre-conditioning aids the efficacy of DNA vaccination by recruiting both Antigen Presenting Cells (APC) and T cells to the vaccine administration site and a more effective activation of the T cells by vaccinated APC.

After pre-conditioning with the chemokine CCL21, a DNA vaccine comprising of VP22-mtGNAQ-PADRE fusion sequence (as illustrated in FIG. 7) may be administered into the chemokine pre-treated site via in vivo electroporation. At least 4 such treatments lead to the activation of the mutation (Q209L)—specific T cells which mediated immunotargeting of the malignant cells harboring Q209L mutation in GNAQ/GNA11 in prophylactic and therapeutic settings, as depicted in FIGS. 10, 11 and 12.

In some embodiments, a DNA vaccine comprising a VP22-mtGNAQ-PADRE fusion sequence could be also used for the ex vivo priming of the T cells as described elsewhere herein. This priming protocol could be used for the activation of mutation-specific T cells ex vivo for the consequent propagation of mutation-specific activated T cells for adoptive transfer of the cells.

In some embodiments, the nucleic acid sequence encoding a fusion protein comprising VP22 or an HLA-binding sequence thereof, a mutant GNAQ sequence comprising a Q209L mutation, and a PADRE epitope is linked with hCCL21 via a P2A ribosome skipping element allowing expression of both transgenes in one cell. In some embodiments, both transgenes are under control of a single promoter. In some embodiments, the promoter is a constitutive promoter. In further embodiments, the promoter is a human elongation factor 1 (EF1) promoter. In some embodiments, the promoter is an inducible promoter.

### Promoters and Constructs

In some embodiments, the nucleic acid sequence encoding a fusion protein comprising VP22 or an HLA-binding sequence thereof, a mutant GNAQ sequence comprising a Q209L mutation, and a PADRE epitope further comprises an operably linked promoter. In some embodiments, the promoter is a constitutive promoter. In further embodiments, the promoter is a human elongation factor 1 (EF1) promoter. In some embodiments, the promoter is an inducible promoter.

In some embodiments, the mutant GNAQ sequence comprising a Q209L mutation is human.

### Pharmaceutical Compositions and Formulations.

The nucleic acid vaccine of the invention may be formulated as a pharmaceutical composition.

Such a pharmaceutical composition may be in a form suitable for administration to a subject (i.e. mammal), or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In one embodiment, the pharmaceutical compositions useful for practicing the method of the invention may comprise an adjuvant. Non-limiting examples of suitable are Freund's complete adjuvant, Freund's incomplete adjuvant, Quil A, Detox, ISCOMs or squalene.

Pharmaceutical compositions that are useful in the methods of the invention may be suitably developed for inhalation, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

The composition of the invention may comprise a preservative from about 0.005% to 2.0% by total weight of the composition. The preservative is used to prevent spoilage in the case of exposure to contaminants in the environment.

### Administration/Dosing

The regimen of administration may affect what constitutes an effective amount. For example, the nucleic acid of the invention may be administered to the subject (i.e. mammal) in a single dose, in several divided dosages, as well as staggered dosages may be administered daily or sequentially, or the dose may be continuously infused, or may be a bolus injection. Further, the dosages may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to treat the disease in the subject. An effective amount of the composition necessary to achieve the intended result will vary and will depend on factors such as the disease to be treated or prevented, the age, sex, weight, condition, general health and prior medical history of the subject being treated, and like factors well-known in the medical arts. In particular embodiments, it is especially advantageous to formulate the composition in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the composition and the heterologous protein to be expressed, and the particular therapeutic effect to be achieved.

### Routes of Administration

One skilled in the art will recognize that although more than one route can be used for administration, a particular route can provide a more immediate and more effective reaction than another route. Routes of administration of any of the compositions\ of the invention include inhalation, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, electroporation and topical administration.

### Kits

In some embodiments a kit is provided for treating, preventing, or ameliorating an a given disease, disorder or condition, or a symptom thereof, as described herein wherein the kit comprises: a) a compound or compositions as described herein; and optionally b) an additional agent or therapy as described herein. The kit can further include instructions or a label for using the kit to treat, prevent, or ameliorate the disease, disorder or condition. In yet other embodiments, the invention extends to kits assays for a given disease, disorder or condition, or a symptom thereof, as described herein. Such kits may, for example, contain the reagents from PCR or other nucleic acid hybridization technology (microarrays) or reagents for immunologically based detection techniques (e.g., ELISpot, ELISA).

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The results of the experiments are now described in the following examples.

### Example 1: Generating and Testing the Vaccine

At The National Center for uveal melanoma at Thomas Jefferson University, about 500 new patients with primary UM are treated at the Wills Eye Hospital (WEH) and about 100 new UM patients with metastasis are treated at the Thomas Jefferson University Hospital (TJUH). There are also no approaches aimed at immune targeting of the mutated GNAQ and GNA11 proteins that are found to be responsible to the tumorigenic transformation of the uveal melanocytes in about 80% of all uveal (ocular) melanomas. Herein is described a new therapeutic treatment and methodologies for treatment of certain cancers. In particular embodiments, the therapeutic is a vaccine designed to activate uveal melanoma-specific immunity.

A challenge in developing UM specific treatments is the lack of animal models mimicking molecular defects and pathology of the UM. To develop appropriate cellular models, non-tumorigenic mouse melanocytes (Melan-a) were stably transduced with a plasmid encoding mutant GNAQ and clones were selected that expressed the mutated protein. Resultant melanocytic cells acquired TPA-independent growth, accelerated cell cycle and ability to produce blue nevus-like lesions after intradermal (id) administration and pulmonary metastases after intravenous (iv) injection (data not shown).

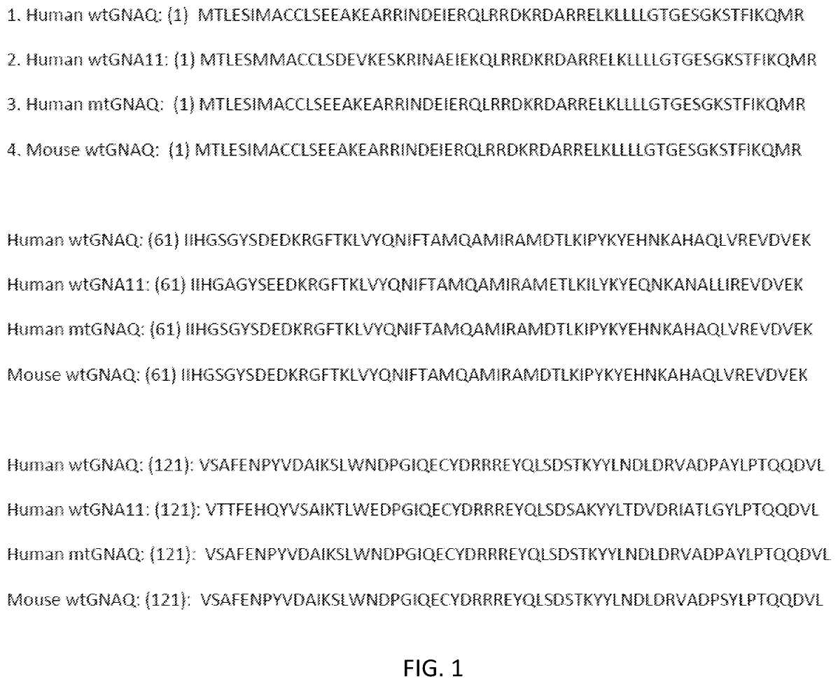

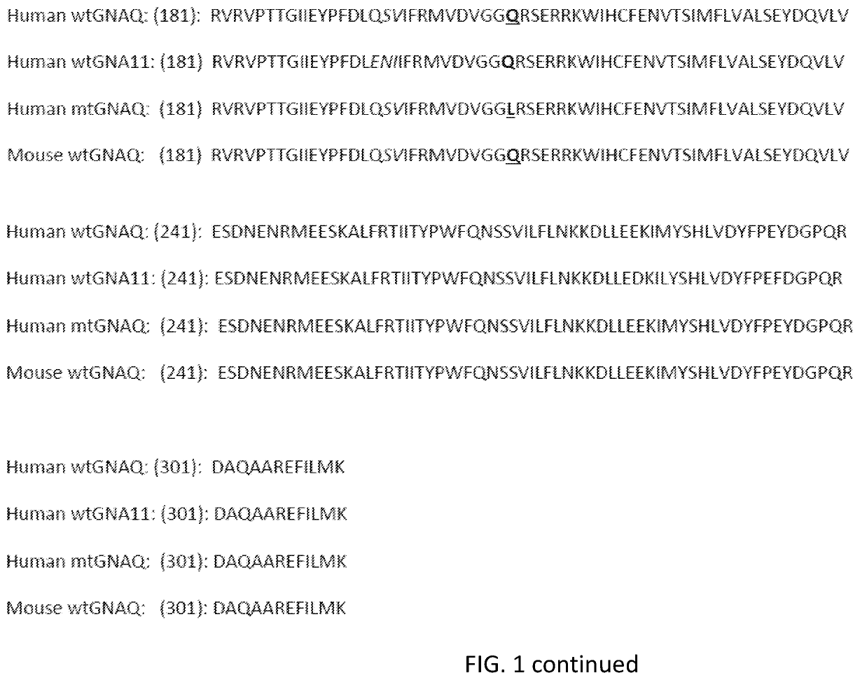

As depicted in FIG. 1, human and mouse GNAQ and GNA11 proteins are highly homologous with 90% identity between two proteins, 99.9% identity between species, and 100% identity in a stretch of 100 amino acids spanning Q209L mutation. Therefore, the mouse model is an adequate, homologous and reliable model to test and develop vaccines and various immuno-therapeutic treatments. FIG. 1 specifies SEQ ID NO: 1, for human wt GNAQ, SEQ ID NO: 2 for human wt GNA11, SEQ ID NO: 3 for human GNAQ containing a Q209L mutation, and SEQ ID NO: 4 for mouse wt GNAQ amino acid sequences. These sequences follow the order from top to bottom in FIG. 1, with the full length sequences running along the several lines in each figure.

In recent years, immunotherapy has emerged as one of the promising modalities to treat various cancers including melanoma. For cutaneous melanoma, immune checkpoint blockade approach showed high clinically relevant outcome after treatment of stage III-IV melanoma patients with antibodies aimed at inhibition of CTLA-4 (ipilimumab) or PD-1. Yet, these approaches exhibited very low response rate in patients with uveal melanoma. Such failure has been attributed to the lack of naturally occurring cytotoxic T cells capable of recognizing and killing this type of malignancy. As there are no methods to induce uveal-melanoma-specific immunity, there is an unmet need to develop vaccination approach that permits generation of cytotoxic T cells targeting uveal melanoma.

Based on the data presented herein, the therapeutic approach disclosed herein induces uveal melanoma-specific immunity. This makes the vaccines widely applicable and cost-effective. In certain embodiments, the therapeutic vaccine could be used alone or in combination with immune checkpoint blockade approach (such as inhibition of CTLA-4 or PD-1).

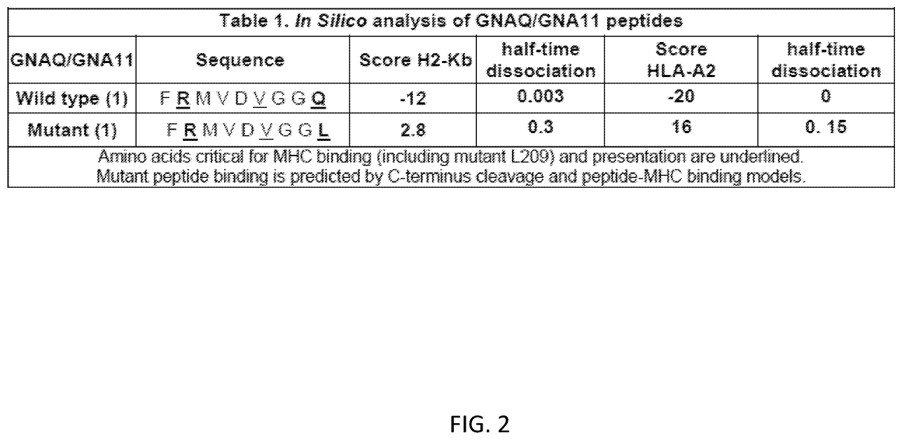

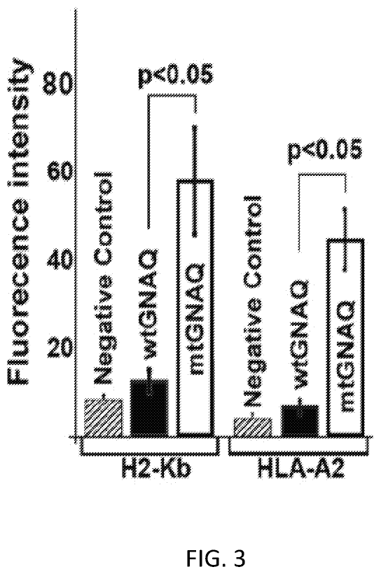

Presentation of the peptides by the class I major histocompatibility complex (MHC) is a prerequisite of the antigen recognition by the immune system. To determine whether an immune system can recognize mutated GNAQ/GNA11 harboring a Q209L substitution, a computerized in silico analysis was conducted of the peptides harboring a Q209L mutation for their ability to bind mouse and human WIC class I. Because more than 50% of uveal melanoma patients are human leukocyte antigen HLA-A2+, this analysis was mostly focused on HLA-A2. As depicted in FIG. 2, computer modeling suggested that Q209L mutated GNAQ peptides have a higher probability of binding to human HLA-A2 than wild type peptides, as reflected by a substantially higher binding probability score and predicted half-time HLA-A2-peptide dissociation. This binding probability was also higher for mouse H2-Kb MHC (FIG. 2). Because mutant L of the identified peptide is located at the P9 position, which is critical for peptide-WIC binding stability, this analysis suggested that T lymphocytes activated against this peptide may discriminate between wild type and mutated GNAQ. This was experimentally confirmed by the in vitro WIC stabilization assay (FIG. 3) showing that Q209L mutated GNAQ peptides unlike wild type counterpart, stabilize both human and mouse MHC on the cell surface.

To further validate immunogenicity of the mutated GNAQ and to determine whether the immune system could be educated to recognize mutated cells in vivo in a mouse animal model, several DNA vaccine vectors were generated encoding mtGNAQ fused in frame with PADRE (mtGNAQ-PADRE) and VP22 epitopes (VP22-mtGNAQ-PADRE) to enhance its immunogenicity. PADRE epitope (Pan human leukocyte antigen-DR reactive epitope) was used in the design of this vaccine because it was shown to activate T helper cells enhancing immunogenicity of vaccines and systemic cytotoxic tumor-antigen-specific cytotoxic response (del Guercio M F, Alexander J, Kubo R T, Arrhenius T, Maewal A, Appella E et al. Potent immunogenic short linear peptide constructs composed of B cell epitopes and Pan DR T helper epitopes (PADRE) for antibody responses in vivo. Vaccine 1997; 15: 441-448; Park J Y, Jin D H, Lee C M, Jang M J, Lee S Y, Shin H S et al. CD4+ TH1 cells generated by Ii-PADRE DNA at prime phase are important to induce effectors and memory CD8b T cells. J Immunother 2010; 33: 510-522). The PADRE epitope sequence is shown in FIG. 4A. Herpes Simplex Virus-derived VP22 was used in the design of the vaccine because it was shown to enhance activation of the cytotoxic T cells after DNA vaccination (Engelhorn, M. E., Guevara-Patino, J. A., Merghoub, T., Liu, C., Ferrone, C. R., Rizzuto, G. A., Cymerman, D. H., Posnett, D. N., Houghton, A. N. and Wolchok, J. D. (2008) Mechanisms of immunization against cancer using chimeric antigens. Molecular therapy: the journal of the American Society of Gene Therapy, 16, 773-781. PMCID: 4399381). The VP22 protein sequence is shown in FIG. 4B. A full-length DNA sequence encoding a VP22-mtGNAQ-PADRE fusion protein is depicted in FIG. 4C. Maps of generated DNA vaccine vectors are depicted in FIGS. 5, 6 and 7. These vaccine vectors contain human elongation factor 1 (EF1) promoter to drive expression of mutant GNAQ with fused immuno-stimulatory polypeptides. A PADRE epitope was inserted at the 3′ end of the mtGNAQ sequence, whereas a VP22-encoding sequence was placed at its 5′ end.

DNA vaccination with the vectors described herein may be performed via in vivo electroporation using intramuscular or intradermal DNA vaccinations as depicted in FIG. 8. Electroporation-based delivery of the DNA vaccine into muscle cells or into the skin results in the expression of the vaccine vector in muscle cells, epidermal keratinocytes, fibroblasts or dermal antigen presenting cells, leading to indirect or direct presentation of the mutated GNAQ peptides and activation of mutant GNAQ-specific T cells (FIG. 8). Without wishing to be bound by theory, co-expression and presentation of the VP22 and PADRE epitopes may additionally enhance activation of the cytotoxic and helper T cells.

Although a full length VP22-encoding sequence was inserted into the vaccine vectors, in silico analysis of the VP22 protein sequence predicted that there are 3 regions within this protein which are enriched with HLA-A1 (amino acids 10-70), HLA-A2 (amino acids 210-260) and HLA-A3(amino acids 167-208) binding peptides as depicted in FIG. 4B and FIG. 9. In FIG. 4B these regions are:
Underlined sequence—region enriched with HLA-A1 binders;
Italicized and underlined sequence—region enriched with HLA-A3 binders
Bolded sequence—region enriched with HLA-A2 binders

FIG. 9 depicts HLA binding peptides. This analysis suggests that the DNA sequences encoding these regions could be used in place of full length VP22 to reduce the size of the vaccine vector (which is usually desirable for DNA vaccines) and to customize vaccines to specific HLA types.

These different therapeutics were then tested in an in vivo DNA vaccination protocol using prophylactic conditions as depicted in FIG. 10. Specifically, 5 cohorts of naïve wild type C57BL6 mice were vaccinated with these DNA vaccine constructs. One cohort also received priming with chemokines as described in our prior studies (Igoucheva, O., Grazzini, M., Pidich, A., Kemp, D., Larijani, M., Farber, M., Lorton, J., Rodeck, U., Alexeev, V.: Immunotargeting and eradication of orthotropic melanoma using a chemokine-enhanced DNA vaccine. Gene Therapy 2013 September; 20(9):939-48.). The first vaccination was designated as day 0 and then additional 3 consecutive vaccinations were performed with one-week intervals (FIG. 10). After a total of 4 vaccinations conducted via intradermal electroporation, activation of the mutant GNAQ-specific T cells was evaluated by IFNγ ELISpot assay. IFNγ ELISpot assay allowed us to detect and enumerate mtGNAQ-specific activated T cells in differently vaccinated mice and compare the efficacy of different vaccines. As depicted in FIG. 11, vaccination with VP22-mtGNAQ-PADRE construct produced the highest number of spot-forming cells, which reflect the number of activated, mtGNAQ-specific, IFNγ-producing T cells.

When comparing IFNγ ELISpot data between all treatment cohorts, it was observed that pre-treatment of the vaccine administration site with chemokine (CCL21) prior to DNA vaccination with VP22-mtGNAQ-PADRE construct produced the highest number of spot-forming cells, that were desired for successful treatment.

Collectively, these studies demonstrated that a DNA vaccine comprised of VP22-mtGNAQ-PADRE was most effective in activating a mtGNAQ-specific T cell response and that priming of the vaccine administration site with CCL21 additionally enhanced T cell activation.

To validate the ability of the DNA vaccination to induce potent T cell-mediated immunity, additional experiments were conducted in therapeutic settings using mice with established pulmonary lesions. As depicted in FIG. 11, intravenous injection of the mtGNAQ malignant cells at day 0 led to the development of the pulmonary experimental metastatic lesions within 2 weeks. These lesions were detected as small pigmented spots in the lungs of sentinel mice. At this time point, a first DNA vaccination (prime immunization) was performed. Vaccinations were continued once a week for 4 weeks. After 6 weeks from mtGNAQ cell intravenous injection, control, Mock treated animals were euthanized. Autopsy confirmed the development of bulky metastatic lesions (FIG. 12, top row). At this time (6 weeks), animals immunized with DNA vaccine encoding mtGNAQ alone showed signs of pulmonary distress and were euthanized one week later (7 weeks). Although pulmonary tumor burden in these animals was lower than in the control cohort, vaccination with mtGNAQ-encoding plasmid was not effective in restricting progression of the neoplastic lesions (FIG. 12, top row). Mice, vaccinated with the mtGNAQ-PADRE vaccine survived for 12 weeks and showed substantially lower tumor burden, whereas animals vaccinated with VP22-mtGNAQ-PADRE vaccine with pre-treatment of the vaccine administration site with the chemokine showed no sign of pulmonary distress for 15 weeks and only few malignant lesions in the lungs (FIG. 12, top row). Accordingly, it is advantageous to provide both the VP-22-mtGNAQ PADRE vaccination alone, or with pre-treatment of the vaccine administration site with the chemokine CCL21.

Activation of mtGNAQ-specific T cell analyzed by IFNγ ELISpot assay correlated well with the observed tumor burden (FIG. 11, lower row). The lowest number of spots detected in wells and, respectively, spot-forming IFNγ-producing activated cells was detected in control, Mock treated mice and in mice vaccinated with mtGNAQ-encoding plasmid, whereas the highest number of spot-forming cells, and, respectively, the highest number of activated T cells, which are desired for effective immunotargeting, was detected in mice vaccinated with VP22-mtGNAQ-PADRE DNA vaccine in conjunction with CCL21 priming of the vaccine administration site. Collectively, these studies showed that vaccination of the tumor-bearing animals with VP22-mtGNAQ-PADRE in conjunction CCL21 led to the most robust mtGNAQ-specific T cell activation and inhibition of metastatic lesions.

Considering that introduction of additional amino acid substitutions into immunogenic peptides could improve its binding to the MHC and enhance T cell activation, in silico analysis of the mutant GNAQ/GNA11 immunogenic peptides was conducted and it was predicted that introduction of the V204P substitution or two substitutions (V205L/E212V) would increase calculated HLA-A2 binding probability (score) and half-time dissociation (FIG. 13). This analysis suggested that these additional substitutions into a DNA vaccine could substantially increase the activity of the DNA vaccine.

To experimentally test whether these additional substitutions could improve mutant GNAQ epitope presentation in the context of human HLA-A2 and T cell activation, corresponding substitutions were introduced into the established VP22-mtGNAQ-PADRE vaccine vector using site-directed mutagenesis and established an ex vivo human T cell priming protocol. This protocol involves isolation of human monocytes and T cells from human peripheral blood, differentiation of antigen presenting cells (APC) from monocytes, ex vivo delivery of DNA vaccine into the APC using in vitro electroporation and co-culture of these “vaccinated” APC with T cells isolated from the same donor.

Using this ex vivo T cell priming protocol, pools of human T cells primed by the APC transduced (“vaccinated”) ex vivo with the original (VP22-mtGNAQ-PADRE) DNA vaccine were generated (FIG. 8) as well as modified vaccine vectors containing additional alterations as depicted in FIG. 13 (V204P (P32) and V205L/E212V (512LV)).

After priming and two re-stimulations of the T cells conducted with 1 week interval, T cells were collected and tested for the ability to target and kill cultured human uveal melanoma cells using various assays. FIGS. 14A-14F illustrate several analyses used for comparison of the mutant GNAQ-specific T cells generated by ex vivo T cell priming. FIGS. 14A and 14B (images of the representative IFNγ ELISpot wells and quantitation of the spot-forming cells, respectively) illustrate superior activation of the mtGNAQ-specific T cells triggered by modified 512LV construct as compared to the original (W32) DNA vaccine design. Assessment of the cytolytic activity of these cells, depicted in FIGS. 14C and 14D, confirmed that (i) the original and the modified DNA vaccines activate mtGNAQ-specific cytotoxic T cell response and that (ii) T cells primed and activated using 512LV construct showed greater CTL activity toward mtGNAQ targets at lower Effector:Target (E:T) ratio. These observations were also confirmed by the T cell tumor binding assay and Granzyme B activity assay (FIGS. 14E, 14F, respectively). Collectively, these data demonstrated that vaccine vector containing additional substitutions such as V205L and E212V (as in the 512LV construct) facilitate activation of the Q209L-specific cytotoxic immune response and, therefore, could be more effective in triggering immune-dedicated targeting and elimination of tumor cells harboring Q209L activating mutation in GNAQ and GNA11 proteins.

Accordingly, the novel therapeutic as described herein comprises a vaccine vector encoding mutated GNAQ with additional co-stimulatory sequences (VP22 and PADRE). In some embodiments, additional substitutions (e.g. 512LV) may be present. The vaccine vectors described herein elicit cytotoxic responses against uveal melanoma harboring an oncogenic (Q209L) mutation in GNAQ and GNA11 proteins.

All in vivo studies were conducted in animal models using BTX830 in vivo electroporation device. In vivo electroporation is one of the most effective means to deliver plasmid DNA into tissues. It involves injection of the DNA in solution (either in water or saline) into a tissue (e.g. skin or muscle) following application of electrodes (e.g. needle electrodes, which are injected into DNA-treated tissue) and application of electric pulses with pre-defined voltage, pulse frequency and length. This approach was widely tested in animal studies (Igoucheva, O., Grazzini, M., Pidich, A., Kemp, D. M., Larijani, M., Farber, M., Lorton, J., Rodeck, U. and Alexeev, V. (2013) Immunotargeting and eradication of orthotopic melanoma using a chemokine-enhanced DNA vaccine. Gene therapy, 20, 939-948.). In vivo electroporation was also successfully used for DNA vaccination in several human clinical trials (e.g. Trimble, C. L., Morrow, M. P., Kraynyak, K. A., Shen, X., Dallas, M., Yan, J., Edwards, L., Parker, R. L., Denny, L., Giffear, M. et al. (2015) Safety, efficacy, and immunogenicity of VGX-3100, a therapeutic synthetic DNA vaccine targeting human papillomavirus 16 and 18 E6 and E7 proteins for cervical intraepithelial neoplasia 2/3: a randomised, double-blind, placebo-controlled phase 2b trial. Lancet, 386, 2078-2088. PMCID: 4888059). DNA vaccination in clinical settings may be performed using intramuscular or intradermal electroporation devices such as FDA-approved Ichor TriGrid (FIG. 15A) or Inovio Cellectra (FIG. 15B) electroporators. Efficacy of DNA vaccination could be improved by pre-treatment of the vaccine administration site with chemokines, such as secondary lymphoid chemokine CCL21, use of a fusion constructs comprising of the VP22 protein or its specific sequences (FIG. 9) and PADRE epitopes, and mutated (Q209L) GNAQ with optional additional substitutions (e.g. 512LV construct, FIG. 13).

### OTHER EMBODIMENTS

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations and subcombinations.

1. A composition comprising a mutant Q209L-GNAQ DNA vaccine encoding, in a N-terminal to C-terminal direction, a fusion protein comprising VP22 or an HLA-binding sequence thereof, a mutant GNAQ sequence comprising a Q209L mutation, and a PADRE epitope.

2. The composition of claim 1, wherein the mutant Q209L-GNAQ sequence comprises additional substitutions selected from the group consisting of V204P and V205L/E212V, wherein the addition substitutions improve binding of the fusion protein to HLA-A2 and enhance T cell activation.

3. (canceled)

4. The composition of claim 1, wherein
(i) the VP 22 is encoded by a nucleic acid sequence having at least 90%, at least 95%, or at least 99% homology to SEQ ID NO: 6 or 18,
(ii) the PADRE epitope is encoded by a nucleic acid sequence having at least 90%, at least 95%, or at least 99% homology to SEQ ID NO: 5, or
(iii) the mutant Q209L-GNAQ DNA vaccine is encoded by a nucleic acid sequence having at least 90%, at least 95%, or at least 99% homology to SEQ ID NO: 12 or 14.

5.-8. (canceled)

9. The composition of claim 1, wherein the mutant GNAQ sequence comprising a Q209L mutation
(i) comprises at least 20 amino acids from Serine at position 198 to Isoleucine at position 217 of SEQ ID NO: 3, or
(ii) is encoded by a nucleic acid sequence comprising SEQ ID NO: 7 or 19.

10. (canceled)

11. A method of treating an ocular cancer having a GNAQ or GNA11 mutation or generating a cytotoxic immune response against uveal melanoma, the method comprising:
administering to a patient a composition comprising a mutant Q209L-GNAQ DNA vaccine encoding, in a N-terminal to C-terminal direction, a fusion protein comprising VP22 or an HLA-binding sequence thereof, a mutant GNAQ sequence comprising a Q209L mutation, and a PADRE epitope.

12. The method of claim 11, wherein
(i) the VP 22 is encoded by a nucleic acid sequence having at least 90%, at least 95%, or at least 99% homology to SEQ ID NO: 6 or 18,
(ii) the PADRE epitope is encoded by a nucleic acid sequence having at least 90%, at least 95%, or at least 99% homology to SEQ ID NO: 5, or
(iii) the mutant Q209L-GNAQ DNA vaccine is encoded by a nucleic acid sequence having at least 90%, at least 95%, or at least 99% homology to SEQ ID NO: 12 or 14.

13. (canceled)

14. (canceled)

15. The method of claim 11, wherein the mutant GNAQ sequence comprising a Q209L mutation
(i) comprises at least 20 amino acids from Serine at position 198 to Isoleucine at position 217 of SEQ ID NO: 3, or
(ii) is encoded by a nucleic acid sequence comprising SEQ ID NO: 7 or 19.

16. (canceled)

17. The method of claim 11, comprising priming of the DNA vaccine administration site with a chemokine.

18.-24. (canceled)

25. A method of providing prophylactic vaccination of a high risk patient after treatment of primary intraocular lesions comprising of malignant cells harboring Q209L mutated GNAQ or GNA11 cells or providing a therapeutic vaccination of a patient with metastatic disease comprising malignant cells harboring Q209L mutated GNAQ or GNA11 cells, said vaccination comprising:
administering to a patient a composition comprising a mutant Q209L-GNAQ DNA vaccine encoding, in a N-terminal to C-terminal direction, a fusion protein comprising VP22 or an HLA-binding sequence thereof, a mutant GNAQ sequence comprising a Q209L mutation, and a PADRE epitope.

26. The method of claim 25, wherein
(i) the VP 22 is encoded by a nucleic acid sequence having at least 90%, at least 95%, or at least 99% homology to SEQ ID NO: 6 or 18,
(ii) the PADRE epitope is encoded by a nucleic acid sequence having at least 90%, at least 95%, or at least 99% homology to SEQ ID NO: 5, or
(iii) the mutant Q209L-GNAQ DNA vaccine is encoded by a nucleic acid sequence having at least 90%, at least 95%, or at least 99% homology to SEQ ID NO: 12 or 14.

27. (canceled)

28. (canceled)

29. The method of claim 25, wherein the mutant GNAQ sequence comprising a Q209L mutation
(i) comprises at least 20 amino acids from Serine at position 198 to Isoleucine at position 217 of SEQ ID NO: 3, or
(ii) is encoded by a nucleic acid sequence comprising SEQ ID NO: 7 or 19.

30. (canceled)

31. The method of claim 25, comprising pre-treating of the vaccine administration site with chemokine CCL21.

32. (canceled)

33. The method of claim 25, wherein said mutant Q209L-GNAQ DNA vaccine encodes a fusion protein wherein the mutant GNAQ sequence comprising a Q209L mutation has at least 90% homology to SEQ ID NO: 3.

34.-44. (canceled)

45. A method of vaccinating a mammal comprising administering to the mammal a composition comprising a mutant Q209L-GNAQ DNA vaccine encoding, in a N-terminal to C-terminal direction, a fusion protein comprising VP22 or an HLA-binding sequence thereof, a mutant GNAQ sequence comprising a Q209L mutation, and a PADRE epitope.

46. The method of claim 45, comprising pre-treatment of a vaccine administration site with CCL21.

47. The method of claim 45, wherein
(i) the VP 22 is encoded by a nucleic acid sequence having at least 90%, at least 95%, or at least 99% homology to SEQ ID NO: 6 or 18,
(ii) the PADRE epitope is encoded by a nucleic acid sequence having at least 90%, at least 95%, or at least 99% homology to SEQ ID NO: 5, or
(iii) the mutant Q209L-GNAQ DNA vaccine is encoded by a nucleic acid sequence having at least 90%, at least 95%, or at least 99% homology to SEQ ID NO: 12 or 14.

48. (canceled)

49. (canceled)

50. The method of claim 45, wherein the mutant GNAQ sequence comprising a Q209L mutation
(i) comprises at least 20 amino acids from Serine at position 198 to Isoleucine at position 217 of SEQ ID NO: 3, or
(ii) is encoded by a nucleic acid sequence comprising SEQ ID NO: 7 or 19.

51. (canceled)

52. A method of creating a vaccine comprising generating a DNA encoding a fusion protein comprising a portion of an antigen and a portion of VP22 comprising regions which are enriched with HLA-A1 (amino acids 10-70), HLA-A2 (amino acids 210-260) and/or HLA-A3(amino acids 167-208) binding peptides of SEQ ID NO: 8.

53. The method of claim 52, wherein the portion of VP22 comprises SEQ ID NO: 8.

54. A method of generating mutant Q209L-GNAQ-specific T cells comprising priming T cells with ex vivo cultured dendritic cells transduced or electroporated with the composition of claim 1.

55. The method of claim 11, wherein said mutant Q209L-GNAQ DNA vaccine encodes a fusion protein wherein the mutant GNAQ sequence comprising a Q209L mutation has at least 90% homology to SEQ ID NO: 3.


SEQUENCE LISTING (26 sequences)

SEQ ID NO: 1 - protein, 312 aa, Artificial Sequence
  Human wt GNAQ
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILMK

SEQ ID NO: 2 - protein, 312 aa, Artificial Sequence
  Human wt GNA11
MTLESMMACCLSDEVKESKRINAEIEKQLRRDKRDARRELKLLLLGTGESGKSTFIKQMRIIHGAGYSEEDKRGFTKLVYQNIFTAMQAMIRAMETLKILYKYEQNKANALLIREVDVEKVTTFEHQYVSAIKTLWEDPGIQECYDRRREYQLSDSAKYYLTDVDRIATLGYLPTQQDVLRVRVPTTGIIEYPFDLENIIFRMVDVGGQRSERRKWIHCFENVTSIMFLVALSEYDQVLVESDNENRMEESKALFRTIITYPWFQNSSVILFLNKKDLLEDKILYSHLVDYFPEFDGPQRDAQAAREFILMK

SEQ ID NO: 3 - protein, 312 aa, Artificial sequence
  Human mtGNAQ
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGLRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILMK

SEQ ID NO: 4 - protein, 312 aa, Artificial Sequence
  Mouse wtGNAQ
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPSYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILMK

SEQ ID NO: 5 - DNA, 39 bp, Artificial Sequence
  PADRE
gcgaaatttgtggcggcgtggaccctgaaagcggcggcg

SEQ ID NO: 6 - DNA, 903 bp, Artificial Sequence
  VP22
atgacctctcgccgctccgtgaagtcgggtccgcgggaggttccgcgcgatgagtacgaggatctgtactacaccccgtcttcaggtatggcgagtcccgatagtccgcctgacacctcccgccgtggcgccctacagacacgctcgcgccagaggggcgaggtccgtttcgtccagtacgacgagtcggattatgccctctacgggggctcgtcatccgaagacgacgaacacccggaggtcccccggacgcggcgtcccgtttccggggcggttttgtccggcccggggcctgcgcgggcgcctccgccacccgctgggtccggaggggccggacgcacacccaccaccgccccccgggccccccgaacccagcgggtggcgactaaggcccccgcggccccggcggcggagaccacccgcggcaggaaatcggcccagccagaatccgccgcactcccagacgcccccgcgtcgacggcgccaacccgatccaagacacccgcgcaggggctggccagaaagctgcactttagcaccgcccccccaaaccccgacgcgccatggaccccccgggtggccggctttaacaagcgcgtcttctgcgccgcggtcgggcgcctggcggccatgcatgcccggatggcggcggtccagctctgggacatgtcgcgtccgcgcacagacgaagacctcaacgaactccttggcatcaccaccatccgcgtgacggtctgcgagggcaaaaacctgcttcagcgcgccaacgagttggtgaatccagacgtggtgcaggacgtcgacgcggccacggcgactcgagggcgttctgcggcgtcgcgccccaccgagcgacctcgagccccagcccgctccgcttctcgccccagacggcccgtcgag

SEQ ID NO: 7 - DNA, 1077 bp, Artificial Sequence
  mtGNAQ
atgactctggagtccatcatggcgtgctgcctgagcgaggaggccaaggaagcccggaggatcaacgacgagatcgagcggcagctgcgcagggacaagcgcgacgcccgccgggagctcaagctgctgctgctggggacaggggagagtggcaagagcaccttcatcaagcagatgaggatcatccacgggtcgggctactctgacgaagacaagcgcggcttcaccaagctggtgtatcagaacatcttcacggccatgcaggccatgatcagagcgatggacacgctcaagatcccatacaagtatgaacacaataaggctcatgcacaattggttcgagaggttgatgtggagaaggtgtctgcttttgagaatccatatgtagatgcaataaagagcttgtggaatgatcctggaatccaggagtgctacgacagacgacgggaatatcagttatctgactctaccaaatactatctgaatgacttggaccgtgtagccgacccttcctatctgcctacacaacaagacgtgcttagagttcgagtccccactacagggatcatcgaatacccctttgacttacaaagtgtcattttcagaatggtcgatgtagggggcctaaggtcagagagaagaaaatggatacactgctttgaaaatgtcacctccatcatgtttctagtagcgcttagcgaatatgatcaagttcttgtggagtcagacaatgagaaccgcatggaggagagcaaagcactctttagaacaattatcacctacccctggttccagaactcctctgtgattctgttcttaaacaagaaagatcttctagaggagaaaatcatgtattcccacctagtcgactacttcccagaatatgatggaccccagagagatgcccaggcagctcgagaattcatcctgaaaatgttcgtggacctgaaccccgacagtgacaaaatcatctactcccacttcacgtgcgccacagataccgagaacatccgcttcgtctttgcagccgtcaaggacaccatcctgcagctgaacctgaaggagtacaatctggtc

SEQ ID NO: 8 - protein, 301 aa, Artificial Sequence
  VP22
MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTQRVATKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVE

SEQ ID NO: 9 - protein, 9 aa, Artificial Sequence
  wt GNAQ/GNA11 peptides
FRMVDVGGQ

SEQ ID NO: 10 - protein, 9 aa, Artificial Sequence
  mtGNAQ/GNA11 peptides
FRMVDVGGL

SEQ ID NO: 11 - protein, 13 aa, Artificial Sequence
  Full length PADRE epitope
AKFVAAWTLKAAA

SEQ ID NO: 12 - DNA, 2040 bp, Artificial Sequence
  Full length VP22-GNAQ-PADRE
gccgccaccatgacctctcgccgctccgtgaagtcgggtccgcgggaggttccgcgcgatgagtacgaggatctgtactacaccccgtcttcaggtatggcgagtcccgatagtccgcctgacacctcccgccgtggcgccctacagacacgctcgcgccagaggggcgaggtccgtttcgtccagtacgacgagtcggattatgccctctacgggggctcgtcatccgaagacgacgaacacccggaggtcccccggacgcggcgtcccgtttccggggcggttttgtccggcccggggcctgcgcgggcgcctccgccacccgctgggtccggaggggccggacgcacacccaccaccgccccccgggccccccgaacccagcgggtggcgactaaggcccccgcggccccggcggcggagaccacccgcggcaggaaatcggcccagccagaatccgccgcactcccagacgcccccgcgtcgacggcgccaacccgatccaagacacccgcgcaggggctggccagaaagctgcactttagcaccgcccccccaaaccccgacgcgccatggaccccccgggtggccggctttaacaagcgcgtcttctgcgccgcggtcgggcgcctggcggccatgcatgcccggatggcggcggtccagctctgggacatgtcgcgtccgcgcacagacgaagacctcaacgaactccttggcatcaccaccatccgcgtgacggtctgcgagggcaaaaacctgcttcagcgcgccaacgagttggtgaatccagacgtggtgcaggacgtcgacgcggccacggcgactcgagggcgttctgcggcgtcgcgccccaccgagcgacctcgagccccagcccgctccgcttctcgccccagacggcccgtcgaggcggccgcaatgactctggagtccatcatggcgtgctgcctgagcgaggaggccaaggaagcccggaggatcaacgacgagatcgagcggcagctgcgcagggacaagcgcgacgcccgccgggagctcaagctgctgctgctggggacaggggagagtggcaagagcaccttcatcaagcagatgaggatcatccacgggtcgggctactctgacgaagacaagcgcggcttcaccaagctggtgtatcagaacatcttcacggccatgcaggccatgatcagagcgatggacacgctcaagatcccatacaagtatgaacacaataaggctcatgcacaattggttcgagaggttgatgtggagaaggtgtctgcttttgagaatccatatgtagatgcaataaagagcttgtggaatgatcctggaatccaggagtgctacgacagacgacgggaatatcagttatctgactctaccaaatactatctgaatgacttggaccgtgtagccgacccttcctatctgcctacacaacaagacgtgcttagagttcgagtccccactacagggatcatcgaatacccctttgacttacaaagtgtcattttcagaatggtcgatgtagggggcctaaggtcagagagaagaaaatggatacactgctttgaaaatgtcacctccatcatgtttctagtagcgcttagcgaatatgatcaagttcttgtggagtcagacaatgagaaccgcatggaggagagcaaagcactctttagaacaattatcacctacccctggttccagaactcctctgtgattctgttcttaaacaagaaagatcttctagaggagaaaatcatgtattcccacctagtcgactacttcccagaatatgatggaccccagagagatgcccaggcagctcgagaattcatcctgaaaatgttcgtggacctgaaccccgacagtgacaaaatcatctactcccacttcacgtgcgccacagataccgagaacatccgcttcgtctttgcagccgtcaaggacaccatcctgcagctgaacctgaaggagtacaatctggtcgcgaaatttgtggcggcgtggaccctgaaagcggcggcgtaa

SEQ ID NO: 13 - protein, 91 aa, Artificial Sequence
  short fusion vaccine
MRLAAMHARMAAVQLWDMSRPRTDEDLNELLGITTIRVTVCEGKNLLQRRANELVNASSVIFRMVDVGGLRSERRKWIAKFVAAWTLKAAA

SEQ ID NO: 14 - DNA, 291 bp, Artificial Sequence
  short fusion vaccine
gcaccatgcgcctggcggccatgcatgcccggatggcggcggtccagctctgggacatgtcgcgtccgcgcacagacgaagacctcaacgaactccttggcatcaccaccatccgcgtgacggtctgcgagggcaaaaacctgcttcagcgcgccaacgagttggtgaatgctagcagtgtcattttcagaatggtcgatgtagggggcctaaggtcagaaagaagaaaatggatagcgaaatttgtggcggcgtggaccctgaaagcggcggcgtaatgatatcgctagc

SEQ ID NO: 15 - protein, 55 aa, Artificial Sequence
  VP22
RLAAMHARMAAVQLWDMSRPRTDEDLNELLGITTIRVTVCEGKNLLQRRANELVN

SEQ ID NO: 16 - protein, 20 aa, Artificial Sequence
  mtGNAQ
SVIFRMVDVGGLRSERRKWI

SEQ ID NO: 17 - protein, 13 aa, Artificial Sequence
  PADRE
AKFVAAWTLKAAA

SEQ ID NO: 18 - DNA, 162 bp, Artificial Sequence
  VP22
cgcctggcggccatgcatgcccggatggcggcggtccagctctgggacatgtcgcgtccgcgcacagacgaagacctcaacgaactccttggcatcaccaccatccgcgtgacggtctgcgagggcaaaaacctgcttcagcgcgccaacgagttggtgaat

SEQ ID NO: 19 - DNA, 60 bp, Artificial Sequence
  mtGNAQ
agtgtcattttcagaatggtcgatgtagggggcctaaggtcagaaagaagaaaatggata

SEQ ID NO: 20 - protein, 9 aa, Artificial Sequence
  Wild type (1) in silico analysis
FRMVDVGGQ

SEQ ID NO: 21 - protein, 9 aa, Artificial Sequence
  Mutant (1) in silico analysis
FRMPDVGGL

SEQ ID NO: 22 - protein, 9 aa, Artificial Sequence
  Wild type (2) in silico analysis
VDVGGQRSE

SEQ ID NO: 23 - protein, 9 aa, Artificial Sequence
  Mutant (2) in silico analysis
VDVGGLRSE

SEQ ID NO: 24 - protein, 9 aa, Artificial Sequence
  V204P
FRMPDVGGL

SEQ ID NO: 25 - protein, 9 aa, Artificial Sequence
  V205L/E212V
VLVGGLRSV

SEQ ID NO: 26 - protein, 9 aa, Artificial Sequence
  Influenza A
GILGFVFTL